(19) United States
(12) Patent Application Publication
(10) Pub. No.: US 2026/0365741 A1
(43) Pub. Date: Nov. 19, 2026

(54) ASSESSING AND TREATING SKIN DISEASE

(71) Applicant: The Johns Hopkins University, Baltimore, MD (US)

(72) Inventors: Shawn Kwatra, Baltimore, MD (US); Martin Prince Alphonse, Baltimore, MD (US)

(21) Appl. No.: 19/126,425

(22) PCT Filed: Nov. 1, 2023
(86) PCT No.: PCT/US2023/036594

§ 371 Date: May 1, 2025

Related U.S. Application Data
(60) Provisional application No. 63/422,061, filed on Nov. 3, 2022.

(51) Int. Cl. C07K 16/28; A61K 31/202; A61K 31/4035; A61K 31/4985; A61K 31/519; A61K 31/52; A61K 31/5377; A61K 31/573; A61K 38/13; A61K 39/00; A61P 17/00; C07K 16/24; G01N 33/68

(52) U.S. Cl. CPC C07K 16/2866; A61K 31/202; A61K 31/4035; A61K 31/4985; A61K 31/519; A61K 31/52; A61K 31/5377; A61K 31/573; A61K 38/13; A61P 17/00; C07K 16/241; C07K 16/244; G01N 33/6869; G01N 33/6881; A61K 2039/507; G01N 2333/54; G01N 2333/5437; G01N 2333/726; G01N 2800/20

(57) ABSTRACT

This document relates to methods and materials for assessing and/or treating mammals (e.g., humans) having an inflammatory skin disease (e.g., erythroderma such as refractory erythroderma). For example, methods and materials that can be used to determine whether or not an inflammatory skin disease (e.g., erythroderma such as refractory erythroderma) is likely to respond to a particular treatment are provided. Methods and materials for treating a mammal (e.g., a human) having an inflammatory skin disease (e.g., erythroderma such as refractory erythroderma) where the treatment is selected based, at least in part, on whether or not the inflammatory skin disease (e.g., erythroderma such as refractory erythroderma) is likely to respond to a particular treatment are also provided.

### CROSS-REFERENCE TO RELATED APPLICATIONS

This application claims priority to U.S. Provisional Patent Application No. 63/422,061, filed on Nov. 3, 2022, which is incorporated herein by reference in its entirety.

### TECHNICAL FIELD

This document relates to methods and materials for assessing and/or treating mammals (e.g., humans) having an inflammatory skin disease (e.g., erythroderma such as refractory erythroderma). For example, methods and materials provided herein can be used to determine whether or not an inflammatory skin disease (e.g., erythroderma such as refractory erythroderma) is likely to respond to a particular treatment. This document also provides methods and materials for treating a mammal (e.g., a human) having an inflammatory skin disease (e.g., erythroderma such as refractory erythroderma) where the treatment is selected based, at least in part, on whether or not the inflammatory skin disease (e.g., erythroderma such as refractory erythroderma) is likely to respond to a particular treatment.

### BACKGROUND INFORMATION

Erythroderma is a heterogeneous systemic disorder characterized by diffuse redness and inflammation involving the majority of body surface area, with an estimated incidence between 1-2 patients per 100,000 (Akhyani et al., BMC Dermatol., 5:5 (2005)). The etiology of erythroderma can include a range of causes, including infection, malignancy, dermatologic conditions, and drug hypersensitivity reactions, making it difficult to predict optimal therapy for patients having this condition (Akhyani et al., BMC Dermatol., 5:5 (2005)).

### SUMMARY

This document provides methods and materials for assessing and/or treating inflammatory skin diseases (e.g., erythroderma such as refractory erythroderma). In some cases, this document provides methods and materials for determining whether or not an inflammatory skin disease (e.g., erythroderma such as refractory erythroderma) is likely to respond to a particular treatment (e.g., a particular targeted therapy). For example, a sample (e.g., a blood sample containing one or more peripheral blood mononuclear cells (PMBCs)) obtained from a mammal (e.g., a human) having an inflammatory skin disease (e.g., erythroderma such as refractory erythroderma) can be assessed to determine if the inflammatory skin disease is likely to respond to a particular treatment (e.g., a particular targeted therapy) based, at least in part, on the immune signature of the inflammatory skin disease. For example, a sample (e.g., a blood sample containing one or more PMBCs) obtained from a mammal (e.g., a human) having an inflammatory skin disease (e.g., erythroderma such as refractory erythroderma) can be assessed for (a) the presence or absence of an elevated level of IL-13 signaling; (b) the presence or absence of an elevated level of IL-17 signaling; and/or (c) the presence or absence of an elevated level of a mas-related G protein-coupled receptor-X2 (MRGPRX2) polypeptide to determine whether or not that inflammatory skin disease (e.g., erythroderma such as refractory erythroderma) is likely to respond to a particular treatment (e.g., a particular targeted therapy). This document also provides methods and materials for treating a mammal (e.g., a human) having an inflammatory skin disease (e.g., erythroderma such as refractory erythroderma) where the treatment is selected based, at least in part, on whether or not the inflammatory skin disease (e.g., erythroderma such as refractory erythroderma) is likely to respond to a particular treatment (e.g., a particular targeted therapy). For example, a mammal (e.g., a human) having an inflammatory skin disease (e.g., erythroderma such as refractory erythroderma) can be administered one or more treatments that are selected based, at least in part, on the immune signature of the inflammatory skin disease (e.g., erythroderma such as refractory erythroderma).

As demonstrated herein, the functional immunophenotype of an inflammatory skin disease (e.g., erythroderma such as refractory erythroderma) can be used to determine whether or not that inflammatory skin disease is likely to respond to a particular treatment. For example, the presence of (a) an elevated level of IL-13 signaling, (b) an elevated level of IL-17 signaling, and (c) an elevated level of a MRGPRX2 polypeptide in PMBCs of a mammal (e.g., a human) having an inflammatory skin disease (e.g., erythroderma such as refractory erythroderma) can be used to identify that mammal as being likely to respond to a particular treatment (e.g., a particular targeted therapy such as one or more inhibitors of the IL-13 signaling pathway (e.g., dupilumab) and/or one or more inhibitors of the IL-17 signaling pathway (e.g., secukinumab)).

Having the ability to identify whether an inflammatory skin disease (e.g., erythroderma such as refractory erythroderma) is likely to respond to a particular treatment (e.g., a particular targeted therapy) as described herein (e.g., based, at least in part, on the immune signature of the inflammatory skin disease (e.g., erythroderma such as refractory erythroderma)) provides a unique and unrealized opportunity to provide an individualized approach for selecting therapies based on the likelihood of the patient response thus providing cost-effective care with better outcomes.

In general, one aspect of this document features methods for assessing an inflammatory skin disease. In some cases, the methods for assessing an inflammatory skin disease can include, or consist essentially of, detecting the presence or absence of: (a) an elevated level of IL-13 signaling, (b) an elevated level of IL-17 signaling, and (c) an elevated level of a MRGPRX2 polypeptide in a sample obtained from a mammal having an inflammatory skin disease; and determining that the mammal is likely to respond to an inhibitor of an IL-13 signaling pathway or an inhibitor of an IL-17 signaling pathway when the presence of at least one of (a), (b), and (c) is detected. In some cases, the methods for assessing an inflammatory skin disease can include, or consist essentially of, detecting the presence or absence of: (a) an elevated level of IL-13 signaling, (b) an elevated level of IL-17 signaling, and (c) an elevated level of a MRGPRX2 polypeptide in a sample obtained from a mammal having an inflammatory skin disease; and determining that the mammal is not likely to respond to said inhibitor of an IL-13 signaling pathway or said inhibitor of an IL-17 signaling pathway when the presence of (a), (b), and (c) is not detected. The mammal can be a human. The inflammatory skin disease can be erythroderma (e.g., refractory erythroderma). The inhibitor of IL-13 signaling pathway can be dupilumab, tralokinumab, lebrikizumab, or eblasakinumab. The inhibitor of IL-17 signaling pathway can be secukinumab.

In another aspect, this document features methods for treating a mammal having an inflammatory skin disease. In some cases, the methods can include, or consist essentially of, determining that a sample obtained from a mammal having an inflammatory skin disease includes the presence of: (a) an elevated level of IL-13 signaling, (b) an elevated level of IL-17 signaling, and (c) an elevated level of a MRGPRX2 polypeptide; and administering an inhibitor of an IL-13 signaling pathway or an inhibitor of an IL-17 signaling pathway to the mammal. In some cases, the methods can include, or consist essentially of, administering an inhibitor of an IL-13 signaling pathway or an inhibitor of an IL-17 signaling pathway to a mammal having an inflammatory skin disease, and identified as having the presence of: (a) an elevated level of IL-13 signaling, (b) an elevated level of IL-17 signaling, and (c) an elevated level of a MRGPRX2 polypeptide. The mammal can be a human. The inflammatory skin disease can be erythroderma (e.g., refractory erythroderma). The inhibitor of IL-13 signaling pathway can be dupilumab, tralokinumab, lebrikizumab, or eblasakinumab. The inhibitor of IL-17 signaling pathway can be secukinumab. The administering can include administering dupilumab and secukinumab to the mammal.

In another aspect, this document features methods for treating a mammal having an inflammatory skin disease. In some cases, the methods can include, or consist essentially of, determining that a sample obtained from a mammal having an inflammatory skin disease lacks the presence of: (a) an elevated level of IL-13 signaling, (b) an elevated level of IL-17 signaling, and (c) an elevated level of a MRGPRX2 polypeptide; and administering an alternative treatment to the mammal, where the alternative treatment is not an inhibitor of an IL-13 signaling pathway or an inhibitor of an IL-17 signaling pathway. In some cases, the methods can include, or consist essentially of, administering an alternative treatment to a mammal having an inflammatory skin disease, and identified as lacking the presence of: (a) an elevated level of IL-13 signaling, (b) an elevated level of IL-17 signaling, and (c) an elevated level of a MRGPRX2 polypeptide, where the alternative treatment is not an inhibitor of an IL-13 signaling pathway or an inhibitor of an IL-17 signaling pathway. The mammal can be a human. The inflammatory skin disease can be erythroderma (e.g., refractory erythroderma). The alternative treatment can include administering prednisone, acitretin, methotrexate, cyclosporine, azathioprine, mycophenolate mofetil, adalimumab, ixekizunab, risakizumab, guselkumab, certolizumab, illumya, ixekizumab, brodalumab, nemolizumab, abrocitinib, upadacitinib, and/or apremilast to the mammal.

In another aspect, this document features uses of a composition comprising an inhibitor of an IL-13 signaling pathway and/or an inhibitor of an IL-17 signaling pathway to treat an inflammatory skin disease.

In another aspect, this document features compositions including an inhibitor of an IL-13 signaling pathway and/or an inhibitor of an IL-17 signaling pathway for use in the preparation of a medicament to treat an inflammatory skin disease.

In another aspect, this document features compositions including an inhibitor of an IL-13 signaling pathway and/or an inhibitor of an IL-17 signaling pathway for use in the treatment of an inflammatory skin disease.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1G. Flow cytometry analysis of Th immune profile and tissue immunofluorescence reveals a unique immune cell phenotype in erythroderma of unknown etiology. FIG. 1A) Clinical presentation of index patient and erythrodermic controls. PRP, pityriasis rubra pilaris; SS, Sezary syndrome. FIG. 1B) PBMC and whole blood flow cytometry study design. FIG. 1C) Representative flow plots of stimulated or unstimulated CD3+ T cells expressing Th cytokines (IL-4, IL-13, IL-17, and IFN-γ). FIG. 1D) Bar plots showing percentages of patient immune cell populations, stratified by percentage of IL-13-producing or IL-17-producing T cells. FIG. 1E) Immunofluorescence co-staining of IL-13 and IL-4R in the lesional skin of the index patient and healthy controls (n=3). FIG. 1F) Immunofluorescence co-staining of IL-17 and IL-17RA in the lesional skin of the index patient and healthy controls. FIG. 1G) Representative flow plots of stimulated or unstimulated CD3+ T cells expressing TGF-β and IL-10 production in index patient and controls.

FIGS. 2A-2B. Gating strategy for cell populations with representative flow plots for the CD3+ T cell population. FIG. 2A) Gating strategy for CD3+ T cell populations; FSC-A, forward scatter area; SSC-A, side scatter area. FIG. 2B) Breakdown of flow cytometry quantification of select cytokines examined in the index patient; IL, interleukin; TGF, tumor growth factor; TNF, tumor necrosis factor; IFN, interferon.

FIGS. 3A-3G. Confirmatory skin staining of select interleukins in the index patient's lesional skin biopsy and healthy controls. FIG. 3A) Hematoxylin and eosin staining of the index patient's skin biopsy. FIG. 3B) Individual and overlaid immunofluorescence staining for IL-13 and IL-4R in the index patient and healthy control; yellow staining in “Merge” demonstrates overlay of IL-13 and IL-4R. FIG. 3C) Quantified fluorescence intensity of IL-13 in the index patient and healthy control. FIG. 3D) Quantified fluorescence intensity of IL-4R in the index patient and healthy control. FIG. 3E) Individual and overlaid immunofluorescence staining for IL-17 and IL-17RA in the index patient and healthy control; yellow staining in “Merge” demonstrates overlay of IL-17RA and IL-17. FIG. 3F) Quantified fluorescence intensity of IL-17 in the index patient and healthy control. FIG. 3F) Quantified Fluorescence intensity of IL-17R in the index patient and healthy control.

FIGS. 4A-4G. Single cell flow cytometric analysis of stimulated T cells reveals distinct immune profiles in various forms of erythroderma. FIG. 2A) Uniform Manifold Approximation and Projection (UMAP) plot of T cell immune populations combined in the index patient, erythroderma controls, and healthy controls. FIG. 2B) UMAP plot of the expression of each individual marker characterizing functional T cell immune populations.

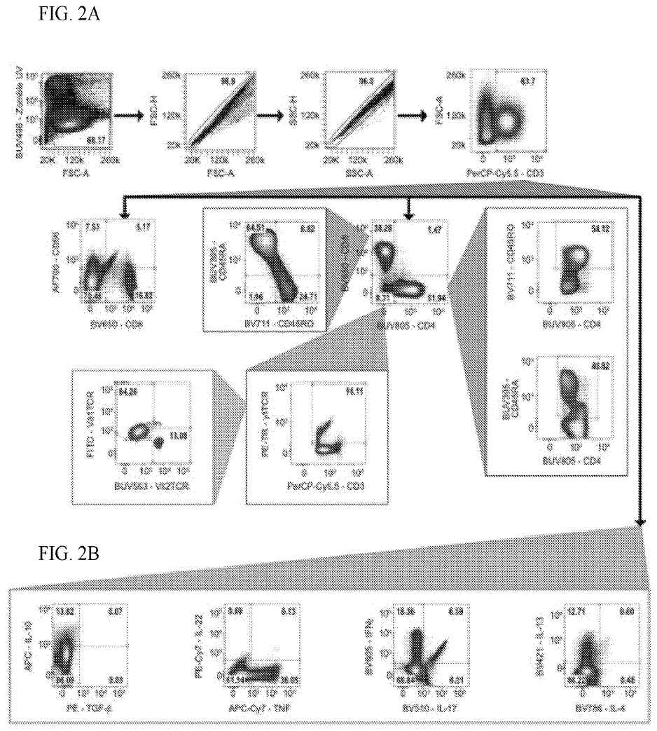

FIG. 2C) UMAP plot of the differences in T cell immune populations between the index patient, erythrodermic controls, and healthy controls. FIG. 2D) Bar plots showing percentages of subtypes of CD3⁺ T cells in the patients. FIG. 2E) Subpopulations of the percentage of CD3⁺ T cell types shown in FIG. 2D. γVδT cells are stratified by γVδ1TCR and γVδ2TCR cells. CD4 T cells are stratified by naive CD4 (CD3⁺CD4⁺CD45RA⁺) and memory CD4 T cells (CD3⁺CD4⁺CD45RO⁺). CD8 T cells are stratified by naive CD8 (CD3⁺CD8⁺CD45RA⁺) and memory CD8 T cells (CD3⁺CD4 CD45RO⁺). FIG. 2F) FlowSOM metaclustering algorithm demonstrating similarities and differences in the T cell immune populations between each group. Each coded color represents an overlay of the immune cell populations and metaclusters. FIG. 2G) Bar plots showing the abundance of the metaclusters between the groups.

FIGS. 5A-5G. Single cell flow cytometric analysis of granulocytic and monocytic immune cell populations from whole blood reveals distinct immune profiles in various forms of erythroderma. FIG. 5A) TriMAP plot of granulocytic and monocytic immune cell populations combined in the index patient, erythroderma controls, and healthy controls. FIG. 5B) TriMAP plot of the expression of each individual marker characterizing whole blood immune populations. FIG. 5C) Uniform Manifold Approximation and Projection (UMAP) plot of the differences in granulocytic and monocytic cell populations between the index patient, erythrodermic controls, and healthy controls. FIG. 5D) Bar plots showing percentages of subtypes of granulocytic and monocytic cell populations in the index patient and controls; DC, dendritic cells. FIG. 5E) Bar plots showing expression of MRGPRX2 and IgE in the index patient and controls; MRGPRX2, Mas-related G-protein coupled receptor member X2; Ig, Immunoglobulin. FIG. 5F) Clustering algorithm identified 95 clusters of immune cells from whole blood: heatmap showing signatures of clusters in the index patient and controls. FIG. 5G) Representative TriMAP plot of clusters with unique immune cell expression patterns in whole blood, including MRGPRX2 and IgE in the index patient.

FIGS. 6A-6H. Gating strategy for whole blood fluorescence-activated cell sorting. FIG. 6A) Gating by CD45, CD14, and CD16 to identify neutrophils and monocytic cells. FIG. 6B) Gating by CD45 and CD11b to identify Siglec-8+ eosinophils and CD163+ macrophages. FIG. 6C) Gating by CD45, FCεR1, and quantification of CD123 (high vs. low) to identify mast cells and basophils. FIG. 6D) Gating by CD62p and CD42b to identify resting and activated platelets. FIG. 6E) Gating by CD45 to identify CD3+ T cells and CD19+ B cells. FIG. 6F) Gating by CD45, CD3, and γδTCR to identify γδT cells; TCR, T cell receptor. FIG. 6G) Identification of MRGPRX2+ cells. FIG. 6H) Identification of IgE+ cells.

FIGS. 7A-7I. Clinical improvement with dual inhibition of IL-4Rα and interleukin-17Rα with reversal of immunopathogenic T cell populations. FIG. 7A) Clinical and treatment course of the index patient. FIG. 7B) TriMAP of all stimulated T cell immune populations in the index patient for all timepoints before and after treatment initiation. FIG. 7C) TriMAP showing changes in stimulated T cell immune populations before and after treatment, stratified by time point. Each time point is represented by a distinct color (TO-pink, T1-blue, T2-green). Differences in immune populations before and after treatment are colored gray. FIG. 7D) TriMAP plot of the expression of each individual marker in stimulated T cells in the index patient. FIG. 7E) Bar plot showing percentages of subtypes of stimulated CD3+ T cells in the index patient at different treatment time points. FIG. 7F) Subpopulations of the percentage of stimulated CD3⁺ T cell types shown in e, γVδT cells are stratified by γVδ1TCR and γVδ2TCR cells. CD4 T cells are stratified by naive CD4 (CD3⁺CD4⁺CD45RA⁺) and memory CD4 T cells (CD3⁺CD4 CD45RO⁺). CD8 T cells are stratified by naive CD8 (CD3⁺CD8⁺CD45RA⁺) and memory CD8 T cells (CD3⁺CD4⁺CD45RO⁺). FIG. 7G) T-distributed stochastic neighbor embedding (tSNE) plot of all unstimulated T cells in the index patient in the three timepoints. FIG. 7H) tSNE plots showing changes in unstimulated T cell immune populations before and after treatment, stratified by time point. Differences in immune populations before and after treatment are colored gray. FIG. 7I) tSNE plots of the expression of each individual marker in unstimulated T cells in the index patient. TO, before treatment, T1 and T2, after treatment.

FIGS. 8A-8D. Changes in clusters in the index patient before and after treatment, including in stimulated (FIGS. 8A and 8B) and unstimulated PBMCs (FIGS. 8C and 8D).

FIGS. 9A-91. Single cell flow cytometric analysis reveals immunopathogenic granulocytic and monocytic immune cell populations with dual inhibition of IL-4Rα and IL-17Rα. FIG. 9A) TriMAP plot of all immune populations in the index patient for all time points before and after treatment initiation. FIG. 9B) TriMAP plot showing changes in immune cell types before and after treatment, stratified by time point. Each time point is represented by a distinct color (TO-pink, T1-blue, T2-green). Differences in immune populations before and after treatment are colored gray. FIG. 9C) TriMAP plot of the expression of each individual marker in the index patient. FIG. 9D) Representative histograms shows the identification of four immune population clusters with breakdown of markers in each cluster. FIG. 9E) Bar plots showing percentages of subtypes of granulocytic and monocytic cell populations in the three timepoints before and after treatment; DC, dendritic cells. FIG. 9F) Illustration of immunopathogenic populations in the whole blood of the index patient, with changes in the makeup of these cells at different timepoints. FIG. 9G) Quantification of mean fluorescence intensity (MFI) and absolute numbers of MRGPRX2-producing cells between different timepoints. FIG. 9H) Quantification of mean fluorescence intensity (MFI) and absolute numbers of IgE-producing cells between different timepoints. FIG. 9I) Bar plots showing expression of MRGPRX2 and IgE at the three timepoints. T0, before treatment, T1 and T2, after treatment.

FIGS. 10A-10C. Improvement of quality of life indices in index patient before and during treatment. T0, before treatment; T1, T2, during treatment. FIG. 10A) Worst-itch numeric rating scale (WI-NRS) score at four time points. FIG. 10B) 5-Dimension (5D) pruritus score at three time points. FIG. 10C) Dermatology Life Quality Index (DLQI) score changes at three time points. T0, before treatment, T1 and T2, after treatment.

### DETAILED DESCRIPTION

This document provides methods and materials that can be used to determine whether or not a mammal (e.g., a human) having an inflammatory skin disease (e.g., erythroderma such as refractory erythroderma) is likely to respond a particular treatment (e.g., a particular targeted therapy). For example, a sample (e.g., a blood sample containing one or more PMBCs) obtained from a mammal (e.g., a human) having an inflammatory skin disease (e.g., erythroderma such as refractory erythroderma) can be assessed for the presence or absence of (a) an elevated level of IL-13 signaling, (b) an elevated level of IL-17 signaling, and/or (c) an elevated level of a MRGPRX2 polypeptide in the sample to determine whether or not the mammal is likely to respond a particular treatment (e.g., a particular targeted therapy). In some cases, the methods and materials provided herein also can include administering one or more treatments (e.g., one or more treatments selected based, at least in part, on whether or not the mammal is likely to respond a particular treatment as described herein) to a mammal having an inflammatory skin disease (e.g., erythroderma such as refractory erythroderma) to treat the mammal.

A mammal (e.g., a human) having an inflammatory skin disease (e.g., erythroderma such as refractory erythroderma) can be assessed to determine whether or not the mammal is likely to respond a particular treatment (e.g., a particular targeted therapy) by detecting the presence or absence of (a) an elevated level of IL-13 signaling, (b) an elevated level of IL-17 signaling, and/or (c) an elevated level of a MRGPRX2 polypeptide in a sample (e.g., a blood sample containing one or more PMBCs) obtained from the mammal. As described herein, the presence of (a) an elevated level of IL-13 signaling, (b) an elevated level of IL-17 signaling, and/or (c) an elevated level of a MRGPRX2 polypeptide in a sample obtained from a mammal having an inflammatory skin disease (e.g., erythroderma such as refractory erythroderma) can be used to determine that the mammal is likely to respond a particular treatment (e.g., a particular targeted therapy).

Any appropriate mammal having an inflammatory skin disease (e.g., erythroderma such as refractory erythroderma) can be assessed and/or treated as described herein. Examples of mammals that can have an inflammatory skin disease (e.g., erythroderma such as refractory erythroderma) and can be assessed and/or treated as described herein include, without limitation, humans, non-human primates (e.g., monkeys), dogs, cats, horses, cows, pigs, sheep, mice, and rats. In some cases, a mammal having an inflammatory skin disease (e.g., erythroderma such as refractory erythroderma) that can be assessed and/or treated as described herein can have one or more conditions associated with inflammatory skin disease. Examples of conditions associated with inflammatory skin disease include, without limitation, psoriasis, contact dermatitis, atopic dermatitis, seborrheic dermatitis, lichen planus, pityriasis rubra pilaris (PRP), Sézary syndrome (SS), hidradenitis suppurativa, drug reactions, dermal hypersensitivity reactions, chronic pruritus of unknown origin, prurigo nodularis, and itchy red bump disease. In some cases, a mammal having an inflammatory skin disease (e.g., erythroderma such as refractory erythroderma) that can be assessed and/or treated as described herein can be using and/or can have been using one or more topical steroids.

When assessing and/or treating a mammal (e.g., a human) having an inflammatory skin disease as described herein, the inflammatory skin disease can be any type of an inflammatory skin disease. An inflammatory skin disease can affect the skin of any part of the body. An inflammatory skin disease can involve any amount of the skin on the body. In some cases, an inflammatory skin disease can involve about 70% or more (e.g., about 70%, about 75%, about 80%, about 85%, about 90%, about 95%, about 98%, or more). Examples of types of inflammatory skin diseases a mammal (e.g., a human) being assessed and/or treated as described herein can have include, without limitation, erythroderma (e.g., erythroderma such as refractory erythroderma), hidradenitis suppurativa, chronic pruritus of unknown origin, prurigo nodularis, atopic dermatitis, psoriasis, lichen planus, dermal hypersensitivity reaction, and itchy red bump disease.

In some cases, the methods described herein can include identifying a mammal (e.g., a human) as having an inflammatory skin disease (e.g., erythroderma such as refractory erythroderma). Any appropriate method can be used to identify a mammal as having an inflammatory skin disease (e.g., erythroderma such as refractory erythroderma). For example, physical examinations can be used to identify a mammal (e.g., a human) as having an inflammatory skin disease (e.g., erythroderma such as refractory erythroderma).

In some cases, a mammal (e.g., a human) having an inflammatory skin disease (e.g., erythroderma such as refractory erythroderma) can be identified as being likely to respond a particular treatment (e.g., particular targeted therapy such as one or more inhibitors of the IL-13 signaling pathway (e.g., dupilumab) and/or one or more inhibitors of the IL-17 signaling pathway (e.g., secukinumab)) based, at least in part, on the presence of an elevated level of IL-13 signaling in a sample (e.g., a blood sample containing one or more PMBCs) obtained from the mammal. The term “elevated level” as used herein with respect to a level of IL-13 signaling in a sample refers to any level that is higher than a reference level of IL-13 signaling. The term “reference level” as used herein with respect to a level of IL-13 signaling refers to the level of IL-13 signaling typically observed in a control sample. Control samples can include, without limitation, samples from one or more mammals that do not have any inflammatory skin disease (e.g., samples from one or more healthy humans). In some cases, an elevated level of IL-13 signaling can be a level that is at least 2 (e.g., at least 5, at least 10, at least 15, at least 20, at least 25, at least 35, or at least 50) fold greater than a reference level of IL-13 signaling. In some cases, an elevated level of IL-13 signaling can be a level that is at least 5% (e.g., at least 10%, at least 15%, at least 20%, at least 25%, or at least 30%) greater than a reference level of IL-13 signaling. It will be appreciated that levels of IL-13 signaling from comparable samples are used when determining whether or not a particular level is an elevated level of polypeptide expression.

Any appropriate method can be used to identify the presence, absence, or level of IL-13 signaling and/or IL-17 signaling. In some cases, an elevated level of a polypeptide involved in IL-13 signaling can be used to identify the presence, absence, or level of IL-13 signaling. For example, an elevated level of an IL-13 polypeptide can be used to identify the presence of an elevated level of IL-13 signaling. For example, an elevated level of an IL-13 polypeptide receptor (e.g., an IL-4R polypeptide) can be used to identify the presence of an elevated level of IL-13 signaling.

In some cases, a mammal (e.g., a human) having an inflammatory skin disease (e.g., erythroderma such as refractory erythroderma) can be identified as being likely to respond a particular treatment (e.g., a particular targeted therapy such as one or more inhibitors of the IL-13 signaling pathway (e.g., dupilumab) and/or one or more inhibitors of the IL-17 signaling pathway (e.g., secukinumab)) based, at least in part, on the presence of an elevated level of IL-17 signaling in a sample (e.g., a blood sample containing one or more PMBCs) obtained from the mammal. The term “elevated level” as used herein with respect to a level of IL-17 signaling in a sample refers to any level that is higher than a reference level of IL-17 signaling. The term “reference level” as used herein with respect to a level of IL-17 signaling refers to the level of IL-17 signaling typically observed in a control sample. Control samples can include, without limitation, samples from one or more mammals that do not have any inflammatory skin disease (e.g., samples from one or more healthy humans). In some cases, an elevated level of IL-17 signaling can be a level that is at least 2 (e.g., at least 5, at least 10, at least 15, at least 20, at least 25, at least 35, or at least 50) fold greater than a reference level of IL-17 signaling. In some cases, an elevated level of IL-17 signaling can be a level that is at least 5% (e.g., at least 10%, at least 15%, at least 20%, at least 25%, or at least 30%) greater than a reference level of IL-17 signaling. It will be appreciated that levels of IL-17 signaling from comparable samples are used when determining whether or not a particular level is an elevated level of polypeptide expression.

Any appropriate method can be used to identify the presence, absence, or level of IL-17 signaling. In some cases, an elevated level of a polypeptide involved in IL-17 signaling can be used to identify the presence, absence, or level of IL-17 signaling. For example, an elevated level of an IL-17 polypeptide can be used to identify the presence of an elevated level of IL-17 signaling. In some cases, an elevated level of an IL-17 polypeptide receptor (e.g., an IL-17Rα polypeptide) can be used to identify the presence of an elevated level of IL-17 signaling.

In some cases, a mammal (e.g., a human) having an inflammatory skin disease (e.g., erythroderma such as refractory erythroderma) can be identified as being likely to respond a particular treatment (e.g., particular targeted therapy such as one or more inhibitors of the IL-13 signaling pathway (e.g., dupilumab) and/or one or more inhibitors of the IL-17 signaling pathway (e.g., secukinumab)) based, at least in part, on the presence of an elevated level of a MRGPRX2 polypeptide in a sample (e.g., a blood sample containing one or more PMBCs) obtained from the mammal. The term “elevated level” as used herein with respect to a level of a MRGPRX2 polypeptide in a sample refers to any level that is higher than a reference level of the MRGPRX2 polypeptide. The term “reference level” as used herein with respect to a level of a MRGPRX2 polypeptide refers to the level of the MRGPRX2 polypeptide typically observed in a control sample. Control samples can include, without limitation, samples from one or more mammals that do not have any inflammatory skin disease (e.g., samples from one or more healthy humans). In some cases, an elevated level of a MRGPRX2 polypeptide can be a level that is at least 2 (e.g., at least 5, at least 10, at least 15, at least 20, at least 25, at least 35, or at least 50) fold greater than a reference level of the MRGPRX2 polypeptide. In some cases, an elevated level of a MRGPRX2 polypeptide can be a level that is at least 5% (e.g., at least 10%, at least 15%, at least 20%, at least 25%, or at least 30%) greater than a reference level of the MRGPRX2 polypeptide. It will be appreciated that levels of a MRGPRX2 polypeptide from comparable samples are used when determining whether or not a particular level is an elevated level of polypeptide expression.

Any appropriate method can be used to identify the presence, absence, or level of a polypeptide (e.g., an IL-13 polypeptide, an IL-4R polypeptide, an IL-17 polypeptide, an IL-17Rα polypeptide, and/or a MRGPRX2 polypeptide). In some cases, the presence, absence, or level of a polypeptide (e.g., an IL-13 polypeptide, an IL-4R polypeptide, an IL-17 polypeptide, an IL-17Rα polypeptide, and/or a MRGPRX2 polypeptide) can be assessed by detecting and/or quantifying mRNA encoding a polypeptide. Examples of methods that can be used to detect and/or quantify mRNA include, without limitation, RT-PCR techniques (e.g., quantitative RT-PCR techniques), and single cell RNASeq. In some cases, the presence, absence, or level of a polypeptide (e.g., an IL-13 polypeptide, an IL-4R polypeptide, an IL-17 polypeptide, an IL-17Rα polypeptide, and/or a MRGPRX2 polypeptide) can be assessed by detecting and/or quantifying the polypeptide. Examples of methods that can be used to detect and/or quantify a polypeptide include, without limitation, immunohistochemistry (IHC) techniques, mass spectrometry techniques (e.g., proteomics-based mass spectrometry assays or targeted quantification-based mass spectrometry assays), western blotting techniques, and flow cytometry. In some cases, the presence, absence, or level of a polypeptide (e.g., an IL-13 polypeptide, an IL-4R polypeptide, an IL-17 polypeptide, an IL-17Rα polypeptide, and/or a MRGPRX2 polypeptide) can be identified as described in Example 1.

In some cases, an elevated level of cells that can produce IL-13 polypeptides and/or IL-17 polypeptides can be used to identify the presence, absence, or level of IL-13 signaling and/or IL-17 signaling. For example, a mammal (e.g., a human) having an inflammatory skin disease (e.g., erythroderma such as refractory erythroderma) can be identified as being likely to respond a particular treatment (e.g., a particular targeted therapy such as one or more inhibitors of the IL-13 signaling pathway (e.g., dupilumab) and/or one or more inhibitors of the IL-17 signaling pathway (e.g., secukinumab)) based, at least in part, on the presence of an elevated level of a population of cells that can produce IL-13 polypeptides and/or IL-17 polypeptides in a sample (e.g., a blood sample containing one or more PMBCs) obtained from the mammal. Examples of cells that can produce IL-13 polypeptides and/or IL-17 polypeptides include, without limitation, gamma delta (γδ) T cells (e.g., γVδ1 T cells and γVδ2 T cells), natural killer (NK) cells, natural killer T (NKT) cells, T memory cells, eosinophils, and mast cells. In some cases, a population of cells that can produce IL-13 polypeptides and/or IL-17 polypeptides can be a clonal population. The term “elevated level” as used herein with respect to a level of cells that can produce IL-13 polypeptides and/or IL-17 polypeptides in a sample refers to any level that is higher than a reference level of those cells. The term “reference level” as used herein with respect to a level of cells that can produce IL-13 polypeptides and/or IL-17 polypeptides refers to the level of those cells typically observed in a control sample. Control samples can include, without limitation, samples from one or more mammals that do not have any inflammatory skin disease (e.g., samples from one or more healthy humans). In some cases, an elevated level of cells that can produce IL-13 polypeptides and/or IL-17 polypeptides can be a level that is at least 2 (e.g., at least 5, at least 10, at least 15, at least 20, at least 25, at least 35, or at least 50) fold greater than a reference level of IL-17 signaling. In some cases, an elevated level of cells that can produce IL-13 polypeptides and/or IL-17 polypeptides can be a level that is at least 5% (e.g., at least 10%, at least 15%, at least 20%, at least 25%, or at least 30%) greater than a reference level of IL-17 signaling. It will be appreciated that levels of cells that can produce IL-13 polypeptides and/or IL-17 polypeptides from comparable samples are used when determining whether or not a particular level is an elevated level of polypeptide expression.

Any appropriate method can be used to identify the presence, absence, or level of cells that can produce IL-13 polypeptides and/or IL-17 polypeptides (e.g., γδT cells such as γVδ1 T cells and γVδ2 T cells). Examples of methods that can be used to detect and/or quantify cells that can produce IL-13 polypeptides and/or IL-17 polypeptides (e.g., γδT cells such as γVδ1 T cells and γVδ2 T cells) include, without limitation, flow cytometry, IHC, and Imaging Mass Cytometry™ (IMC™). In some cases, the presence, absence, or level of a polypeptide (e.g., an IL-13 polypeptide, an IL-4R polypeptide, an IL-17 polypeptide, and/or an IL-17Rα polypeptide) can be identified as described in Example 1.

Any appropriate sample from a mammal (e.g., a human) having an inflammatory skin disease (e.g., erythroderma such as refractory erythroderma) can be assessed as described herein (e.g., to determine whether or not the mammal is likely to respond a particular treatment based, at least in part, on the presence or absence of (a) an elevated level of IL-13 signaling, (b) an elevated level of IL-17 signaling, and/or (c) an elevated level of a MRGPRX2 polypeptide in a sample obtained from the mammal). In some cases, a sample can be a biological sample. In some cases, a sample can contain one or more PMBCs (e.g., stimulated PBMCs or unstimulated PBMCs). In some cases, a sample can contain one or more biological molecules (e.g., nucleic acids such as DNA and RNA, polypeptides, carbohydrates, lipids, hormones, and/or metabolites). Examples of samples that can be assessed as described herein include, without limitation, fluid samples (e.g., whole blood, serum, plasma, urine, and saliva), tissue samples (e.g., skin, tissue samples), and cellular samples (e.g., buccal swabs). In some cases, one or more biological molecules can be isolated from a sample. For example, nucleic acid can be isolated from a sample and can be assessed as described herein. For example, polypeptides can be isolated from a sample and can be assessed as described herein. A sample can be obtained using any appropriate method. For example, when a sample is a skin sample, a tape strip can be used to obtain the skin sample.

In some cases, a mammal (e.g., a human) having an inflammatory skin disease (e.g., erythroderma such as refractory erythroderma) and identified as being likely to respond a particular treatment (e.g., a particular targeted therapy) as described herein (e.g., based, at least in part, on the presence of (a) an elevated level of IL-13 signaling, (b) an elevated level of IL-17 signaling, and/or (c) an elevated level of a MRGPRX2 polypeptide) can be selected for treatment with one or more immunotherapies (e.g., one or more immune checkpoint inhibitors). For example, a mammal having an inflammatory skin disease (e.g., erythroderma such as refractory erythroderma) and identified as having the presence of (a) an elevated level of IL-13 signaling, (b) an elevated level of IL-17 signaling, and/or (c) an elevated level of a MRGPRX2 polypeptide in a sample obtained from the mammal can be selected to receive one or more (e.g., one, two, three, four, five, or more) particular targeted therapies such as one or more inhibitors of the IL-13 signaling pathway (e.g., dupilumab) and/or one or more inhibitors of the IL-17 signaling pathway (e.g., secukinumab).

In some cases, a mammal (e.g., a human) having an inflammatory skin disease (e.g., erythroderma such as refractory erythroderma) and identified as not being likely to respond a particular treatment (e.g., a particular targeted therapy) as described herein (e.g., based, at least in part, on the absence of (a) an elevated level of IL-13 signaling, (b) an elevated level of IL-17 signaling, and (c) an elevated level of a MRGPRX2 polypeptide) can be selected for treatment with one or more alternative treatments (e.g., one or more treatments that are not an inhibitor of the IL-13 signaling pathway (e.g., dupilumab) or an inhibitor of the IL-17 signaling pathway (e.g., secukinumab)). For example, a mammal having an inflammatory skin disease (e.g., erythroderma such as refractory erythroderma) and identified as lacking (a) an elevated level of IL-13 signaling, (b) an elevated level of IL-17 signaling, and (c) an elevated level of a MRGPRX2 polypeptide in a sample obtained from the mammal can be selected to receive one or more (e.g., one, two, three, four, five, or more) alternative treatments.

This document also provides methods for treating a mammal (e.g., a human) having an inflammatory skin disease (e.g., erythroderma such as refractory erythroderma). In some cases, a mammal (e.g., a human) having an inflammatory skin disease (e.g., erythroderma such as refractory erythroderma) and assessed as described herein (e.g., to determine whether or not the mammal is likely to respond a particular treatment based, at least in part, on the presence or absence of (a) an elevated level of IL-13 signaling, (b) an elevated level of IL-17 signaling, and/or (c) an elevated level of a MRGPRX2 polypeptide in a sample obtained from the mammal) can be administered or instructed to self-administer one or more (e.g., one, two, three, four, five, or more) treatments, where the one or more treatments are effective to treat the inflammatory skin disease (e.g., erythroderma such as refractory erythroderma). For example, a mammal having an inflammatory skin disease (e.g., erythroderma such as refractory erythroderma) can be administered or instructed to self-administer one or more treatments selected based, at least in part, on whether or not the mammal is likely to respond a particular treatment (e.g., a particular targeted therapy such as one or more inhibitors of the IL-13 signaling pathway (e.g., dupilumab) and/or one or more inhibitors of the IL-17 signaling pathway (e.g., secukinumab) based, at least in part, on the presence or absence of (a) an elevated level of IL-13 signaling, (b) an elevated level of IL-17 signaling, and/or (c) an elevated level of a MRGPRX2 polypeptide in a sample obtained from the mammal.

In general, a treatment for an inflammatory skin disease (e.g., erythroderma such as refractory erythroderma) can include any appropriate inflammatory skin disease treatment. In some cases, an inflammatory skin disease (e.g., erythroderma such as refractory erythroderma) treatment can include administering steroids (e.g., topical steroids), retinoids, immunosuppressive agents, antihistamines (e.g., sedative antihistamines), and/or antimicrobial agents to a mammal in need thereof. Examples of inflammatory skin disease treatments include, without limitation, prednisone, acitretin, methotrexate, cyclosporine, azathioprine, mycophenolate mofetil, adalimumab, ixekizunab, risakizumab, guselkumab, and certolizumab.

When treating a mammal (e.g., a human) having an inflammatory skin disease (e.g., erythroderma such as refractory erythroderma) and identified as being likely to respond a particular treatment (e.g., a particular targeted therapy) as described herein (e.g., based, at least in part, on the presence of (a) an elevated level of IL-13 signaling, (b) an elevated level of IL-17 signaling, and/or (c) an elevated level of a MRGPRX2 polypeptide), the mammal can be administered or instructed to self-administer one or more (e.g., one, two, three, four, five, or more) particular targeted therapies such as one or more inhibitors of the IL-13 signaling pathway (e.g., dupilumab) and/or one or more inhibitors of the IL-17 signaling pathway (e.g., secukinumab). For example, a mammal having an inflammatory skin disease (e.g., erythroderma such as refractory erythroderma) and identified as having the presence of (a) an elevated level of IL-13 signaling, (b) an elevated level of IL-17 signaling, and/or (c) an elevated level of a MRGPRX2 polypeptide in a sample obtained from the mammal can be administered or instructed to self-administer one or more inhibitors of IL-13 signaling and/or one or more inhibitors of IL-17 signaling.

An inhibitor of IL-13 signaling can be any appropriate inhibitor of IL-13 signaling. In some cases, an inhibitor of IL-13 signaling can be a molecule that reduces or eliminates the signaling between an IL-13 polypeptide its receptor (e.g., an IL-4R polypeptide). In some cases, an inhibitor of IL-13 signaling can be an inhibitor of an IL-13 polypeptide (e.g., an inhibitor of IL-13 polypeptide expression or an inhibitor of IL-13 polypeptide activity). In some cases, an inhibitor of IL-13 signaling can be an inhibitor of an IL-4R polypeptide (e.g., an inhibitor of IL-4R polypeptide expression or an inhibitor of IL-4R polypeptide activity). Examples of compounds that can reduce or eliminate polypeptide activity include, without limitation, antibodies (e.g., neutralizing antibodies), and small molecules. Examples of compounds that can reduce or eliminate polypeptide expression include, without limitation, nucleic acid molecules designed to induce RNA interference (e.g., a siRNA molecule or a shRNA molecule), antisense molecules, and miRNAs. Examples of inhibitors of IL-13 signaling include without limitation, dupilumab (e.g., DUPIXENT®), tralokinumab, lebrikizumab, and eblasakinumab.

An inhibitor of IL-17 signaling can be any appropriate inhibitor of IL-17 signaling. In some cases, an inhibitor of IL-17 signaling a molecule that reduces or eliminates the signaling between an IL-17 polypeptide its receptor (e.g., an IL-17Rα polypeptide). In some cases, an inhibitor of IL-17 signaling can be an inhibitor of an IL-17 polypeptide (e.g., an inhibitor of IL-17 polypeptide expression or an inhibitor of IL-17 polypeptide activity). In some cases, an inhibitor of IL-17 signaling can be an inhibitor of an IL-17Rα polypeptide (e.g., an inhibitor of IL-17Rα polypeptide expression or an inhibitor of IL-17Rα polypeptide activity). Examples of compounds that can reduce or eliminate polypeptide activity include, without limitation, antibodies (e.g., neutralizing antibodies), and small molecules. Examples of compounds that can reduce or eliminate polypeptide expression include, without limitation, nucleic acid molecules designed to induce RNA interference (e.g., a siRNA molecule or a shRNA molecule), antisense molecules, and miRNAs. An example of an inhibitor of IL-17 signaling includes, without limitation, secukinumab (e.g., COSENTYX®)

In some cases, when treating a mammal (e.g., a human) having an inflammatory skin disease (e.g., erythroderma such as refractory erythroderma) and identified as being likely to respond a particular treatment (e.g., a particular targeted therapy such as one or more inhibitors of the IL-13 signaling pathway (e.g., dupilumab) and/or one or more inhibitors of the IL-17 signaling pathway (e.g., secukinumab)) as described herein (e.g., based, at least in part, on the presence of (a) an elevated level of IL-13 signaling, (b) an elevated level of IL-17 signaling, and/or (c) an elevated level of a MRGPRX2 polypeptide) the one or more inhibitors of the IL-13 signaling pathway (e.g., dupilumab) and/or one or more inhibitors of the IL-17 signaling pathway (e.g., secukinumab) can be the sole active agent(s) administered to the mammal to treat the inflammatory skin disease.

In some cases, when treating a mammal (e.g., a human) having an inflammatory skin disease (e.g., erythroderma such as refractory erythroderma) and identified as being likely to respond a particular treatment (e.g., a particular targeted therapy such as one or more inhibitors of the IL-13 signaling pathway (e.g., dupilumab) and/or one or more inhibitors of the IL-17 signaling pathway (e.g., secukinumab)) as described herein (e.g., based, at least in part, on the presence of (a) an elevated level of IL-13 signaling, (b) an elevated level of IL-17 signaling, and/or (c) an elevated level of a MRGPRX2 polypeptide) the one or more inhibitors of the IL-13 signaling pathway (e.g., dupilumab) and/or one or more inhibitors of the IL-17 signaling pathway (e.g., secukinumab) can be administered to the mammal together with one or more additional agents/therapies used to treat an inflammatory skin disease. In some cases, an agent that can be administered to the mammal together with one or more additional agents/therapies used to treat an inflammatory skin disease (e.g., erythroderma such as refractory erythroderma) can be a steroid (e.g., a topical steroid). In some cases, an agent that can be administered to the mammal together with one or more additional agents/therapies used to treat an inflammatory skin disease (e.g., erythroderma such as refractory erythroderma) can be a retinoid. In some cases, an agent that can be administered to the mammal together with one or more additional agents/therapies used to treat an inflammatory skin disease (e.g., erythroderma such as refractory erythroderma) can be an antihistamine (e.g., a sedative antihistamine). In some cases, an agent that can be administered to the mammal together with one or more additional agents/therapies used to treat an inflammatory skin disease (e.g., erythroderma such as refractory erythroderma) can be an antimicrobial agent. Examples of treatments that can be administered to a mammal (e.g., a human) having an inflammatory skin disease (e.g., erythroderma such as refractory erythroderma) and together with one or more inhibitors of the IL-13 signaling pathway (e.g., dupilumab) and/or one or more inhibitors of the IL-17 signaling pathway (e.g., secukinumab) include, without limitation, prednisone, acitretin, methotrexate, cyclosporine, azathioprine, mycophenolate mofetil, adalimumab, ixekizunab, risakizumab, guselkumab, and certolizumab, and any combinations thereof. In cases where one or more inhibitors of the IL-13 signaling pathway (e.g., dupilumab) and/or one or more inhibitors of the IL-17 signaling pathway (e.g., secukinumab) are used in combination with additional agents used to treat an inflammatory skin disease (e.g., erythroderma such as refractory erythroderma), the one or more additional agents can be administered at the same time (e.g., in a single composition containing both one or more inhibitors of the IL-13 signaling pathway (e.g., dupilumab) and/or one or more inhibitors of the IL-17 signaling pathway (e.g., secukinumab) and the one or more additional agents) or independently. For example, one or more inhibitors of the IL-13 signaling pathway (e.g., dupilumab) and/or one or more inhibitors of the IL-17 signaling pathway (e.g., secukinumab) can be administered first, and the one or more additional agents administered second, or vice versa. Examples of therapies that can be used to treat an inflammatory skin disease (e.g., erythroderma such as refractory erythroderma) include, without limitation, phototherapies such as extracorporeal photophoresis. In cases where one or more inhibitors of the IL-13 signaling pathway (e.g., dupilumab) and/or one or more inhibitors of the IL-17 signaling pathway (e.g., secukinumab) are used in combination with one or more additional therapies used to treat an inflammatory skin disease (e.g., erythroderma such as refractory erythroderma), the one or more additional therapies can be performed at the same time or independently of the administration of the one or more inhibitors of the IL-13 signaling pathway (e.g., dupilumab) and/or one or more inhibitors of the IL-17 signaling pathway (e.g., secukinumab). For example, one or more inhibitors of the IL-13 signaling pathway (e.g., dupilumab) and/or one or more inhibitors of the IL-17 signaling pathway (e.g., secukinumab) can be administered before, during, or after the one or more additional therapies are performed.

When treating a mammal (e.g., a human) having an inflammatory skin disease (e.g., erythroderma such as refractory erythroderma) and identified as not being likely to respond a particular treatment (e.g., a particular targeted therapy) as described herein (e.g., based, at least in part, on the absence of an elevated level of IL-13 signaling and the absence of an elevated level of IL-17 signaling), the mammal can be administered or instructed to self-administer one or more alternative treatments (e.g., one or more treatments that are not an inhibitor of the IL-13 signaling pathway (e.g., dupilumab) or an inhibitor of the IL-17 signaling pathway (e.g., secukinumab)). For example, a mammal having an inflammatory skin disease (e.g., erythroderma such as refractory erythroderma) and identified as lacking the presence of (a) an elevated level of IL-13 signaling, (b) an elevated level of IL-17 signaling, and (c) an elevated level of a MRGPRX2 polypeptide in a sample obtained from the mammal can be administered or instructed to self-administer one or more alternative treatments. In some cases, an alternative treatment that can be used to treat an inflammatory skin disease (e.g., erythroderma such as refractory erythroderma) can be a retinoid. In some cases, an alternative treatment that can be used to treat an inflammatory skin disease (e.g., erythroderma such as refractory erythroderma) can be an antihistamine (e.g., a sedative antihistamine). In some cases, an alternative treatment that can be used to treat an inflammatory skin disease (e.g., erythroderma such as refractory erythroderma) can be an antimicrobial agent. In some cases, an alternative treatment that can be used to treat an inflammatory skin disease (e.g., erythroderma such as refractory erythroderma) can include subjecting the mammal to one or more phototherapies (e.g., extracorporeal photophoresis). In some cases, an alternative treatment that can be used to treat an inflammatory skin disease (e.g., erythroderma such as refractory erythroderma) can be an inflammatory skin disease treatment that is not an inhibitor of the IL-13 signaling pathway and it not an inhibitor of the IL-17 signaling pathway. Examples of alternative treatments that can be administered to a mammal (e.g., a human) having an inflammatory skin disease (e.g., erythroderma such as refractory erythroderma) and lacking the presence of (a) an elevated level of IL-13 signaling, (b) an elevated level of IL-17 signaling, and (c) an elevated level of a MRGPRX2 polypeptide in a sample obtained from the mammal to treat the mammal include, without limitation, prednisone, acitretin, methotrexate, cyclosporine, azathioprine, mycophenolate mofetil, adalimumab, ixekizunab, risakizumab, guselkumab, certolizumab, illumya, ixekizumab, brodalumab, nemolizumab, abrocitinib, upadacitinib, apremilast, and combinations thereof.

In some cases, a mammal (e.g., a human) having an inflammatory skin disease (e.g., erythroderma such as refractory erythroderma) and identified as not being likely to respond a particular treatment (e.g., a particular targeted therapy) as described herein (e.g., based, at least in part, on the absence of an elevated level of IL-13 signaling and the absence of an elevated level of IL-17 signaling) is not administered an inhibitor of the IL-13 signaling pathway (e.g., dupilumab) or an inhibitor of the IL-17 signaling pathway (e.g., secukinumab).

In some cases, when treating a mammal (e.g., a human) having an inflammatory skin disease (e.g., erythroderma such as refractory erythroderma) as described herein, the treatment can be effective to reduce or eliminate the amount of body surface affected by the inflammatory skin disease. For example, the methods and materials described herein can be used to reduce the amount of body surface of a mammal affected by an inflammatory skin disease (e.g., erythroderma such as refractory erythroderma) by, for example, 10, 20, 30, 40, 50, 60, 70, 80, 90, 95, or more percent.

In some cases, when treating a mammal (e.g., a human) having an inflammatory skin disease (e.g., erythroderma such as refractory erythroderma) as described herein, the treatment can be effective to reduce or eliminate one or more symptoms of the inflammatory skin disease. Examples of symptoms of an inflammatory skin disease (e.g., erythroderma such as refractory erythroderma) that can be reduced or eliminated using the methods and materials described herein can include, without limitation, pruritus (itchy skin), skin redness, skin inflammation, and skin pain. For example, the methods and materials described herein can be used to reduce one or more symptoms of an inflammatory skin disease (e.g., erythroderma such as refractory erythroderma) by, for example, 10, 20, 30, 40, 50, 60, 70, 80, 90, 95, or more percent.

In some cases, when treating a mammal (e.g., a human) having an inflammatory skin disease (e.g., erythroderma such as refractory erythroderma) as described herein, the treatment can be effective to reduce or eliminate one or more complications associated with the inflammatory skin disease. Examples of complications of an inflammatory skin disease (e.g., erythroderma such as refractory erythroderma) that can be reduced or eliminated using the methods and materials described herein include, without limitation, heat loss (e.g., hypothermia), fluid loss (e.g., electrolyte abnormalities and dehydration), secondary skin infections (e.g., impetigo and cellulitis), anxiety, depression, sleep disturbance, pruritus, and pain. For example, the methods and materials described herein can be used to reduce one or more complications associated with an inflammatory skin disease (e.g., erythroderma such as refractory erythroderma) by, for example, 10, 20, 30, 40, 50, 60, 70, 80, 90, 95, or more percent.

In some cases, a course of treatment, the severity of one or more symptoms related to an inflammatory skin disease (e.g., erythroderma such as refractory erythroderma) can be monitored.

The invention will be further described in the following examples, which do not limit the scope of the invention described in the claims.

### EXAMPLES

### Example 1: Targeted IL-13 and IL-17-Directed Therapy Guided by Single Cell Analysis in Erythroderma of Unknown Etiology

Erythroderma is a systemic heterogeneous disorder characterized by diffuse redness and inflammation involving the majority of the skin's body surface area.

This Example describes the identification of dysregulation of both Th2 and Th17 immune responses with increased interleukin (IL)-13 and IL-17 cytokines in erythroderma.

### Results

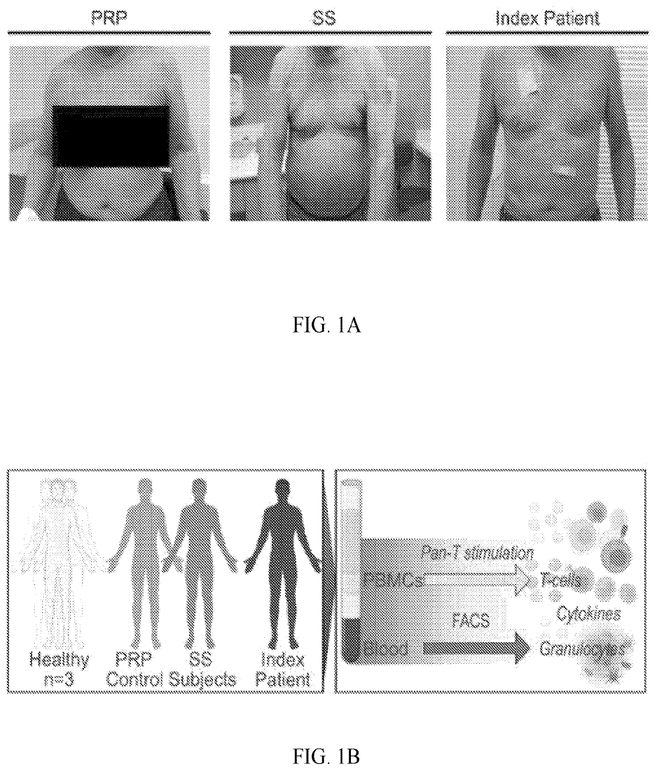

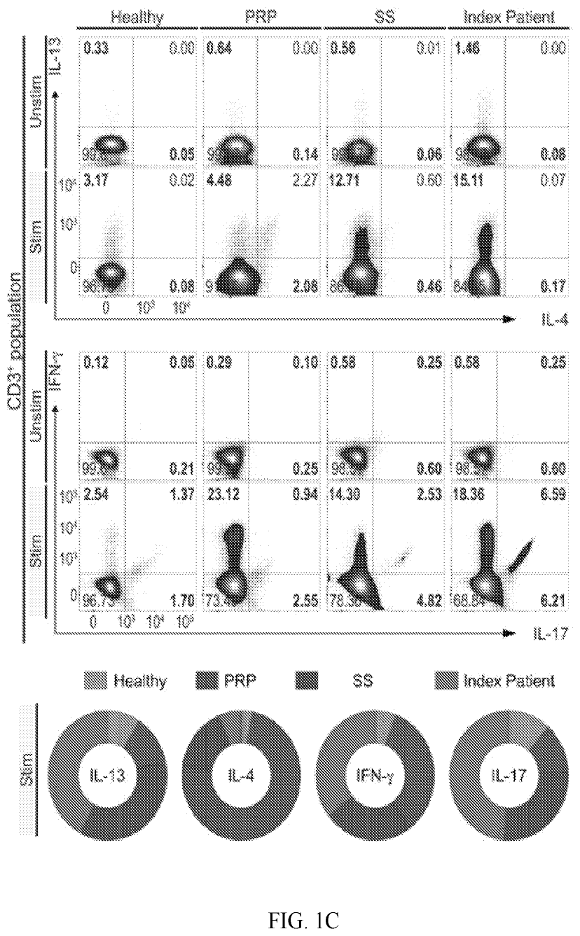

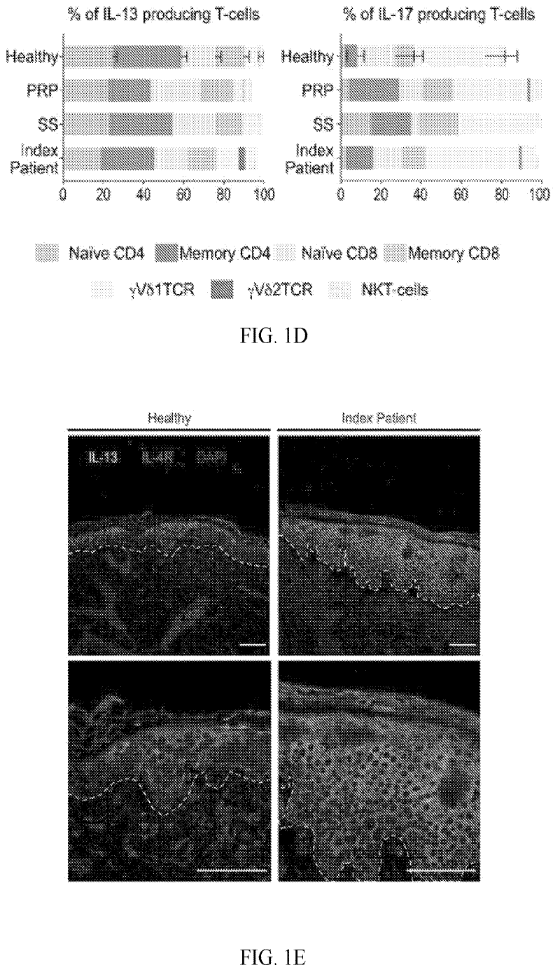

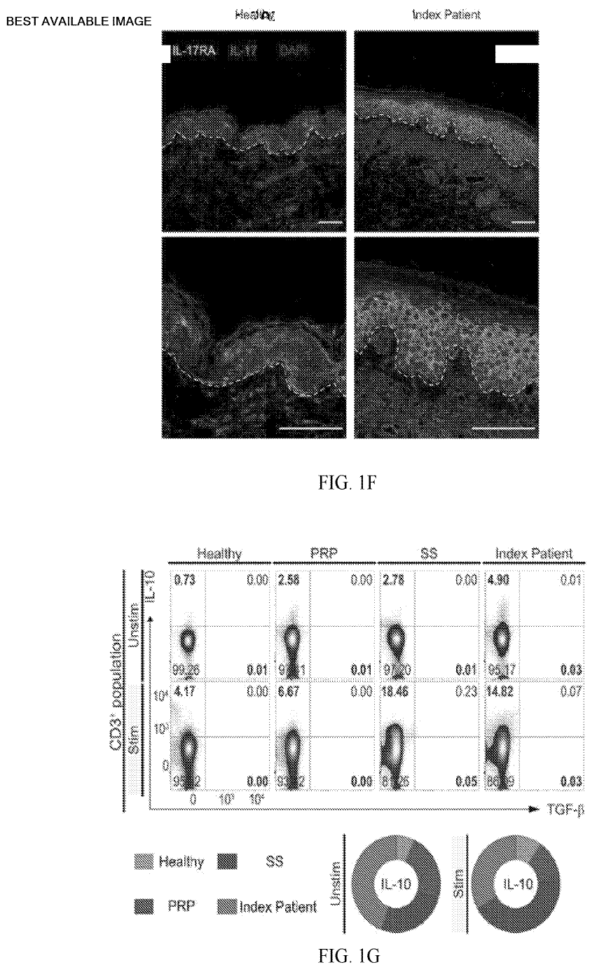

To identify immunopathogenic populations and immune cell composition in the index patient, functional immunophenotyping was performed in the index patient and compared with two erythrodermic controls, one with PRP and the other with SS (FIG. 1A) along with healthy controls (n=3). PRP and SS were selected as the control conditions, as these patients presented with diffuse erythroderma, a known manifestation of these conditions. PBMCs were stimulated with a pan-T stimulation (phorbol 12-myristate 13-acetate (PMA)-ionomycin), and T cell differentiation was determined by fluorescence-activated cell sorting (FACS). To further examine the granulocytic population, FACS was performed on the patients' whole blood. The schematic outline is shown in FIG. 1B.

a Combined Th2 and Th17 Immune Profiles were Present in the Index Patient.

To characterize T cell differentiation, the PBMCs were stimulated with a non-specific pan-T cell stimulation (PMA+ionomycin) and the Th immune profile was assessed by evaluating the intracellular interleukin (IL)-4, IL-13, IL-17, and interferon (IFN)-γ cytokines in both stimulated and unstimulated CD3⁺ T cells from the index patient and from erythrodermic and healthy controls. The gating strategy for flow cytometry of the CD3⁺ T cell populations is depicted in FIG. 2. In the index patient, stimulated T cells expressed more IL-13 (15.11%) and IL-17 (6.21%) cytokines when compared to healthy controls (3.17% and 1.70% for IL-13 and IL-17, respectively), the PRP patient (4.48% and 2.55% for IL-13 and IL-17, respectively), and the SS patient (12.71% and 4.82% for IL-13 and IL-17, respectively) (p<0.05 for all comparisons; FIG. 1C). When stratified by types of CD3⁺ IL-13-secreting cells, the index patient had an elevated population of the clonal γVδ2 T cells when compared to healthy or erythrodermic controls. Additionally, among the stratified types of CD3⁺ IL-17 producing cells, the index patient had the highest level of clonal γVδ1 T cells and NK-T cells compared to all other patient populations examined, and only the index patient and PRP patients demonstrated any clonal γVδ2 T cells. (FIG. 1D).

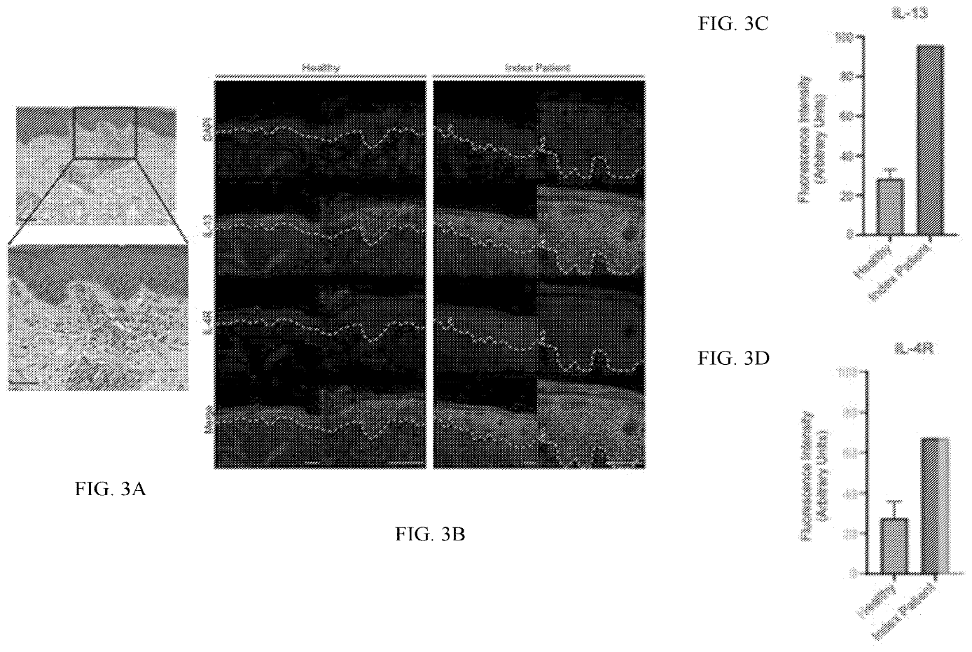

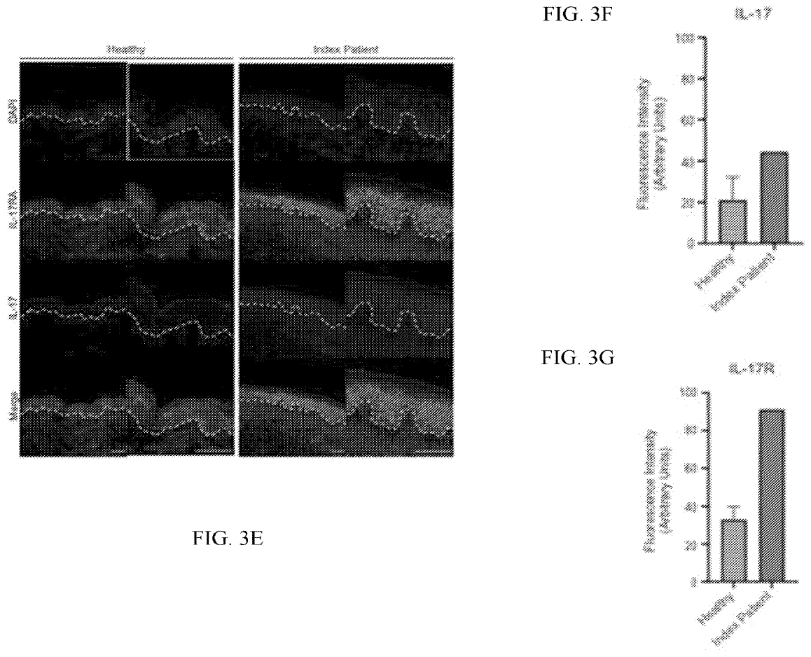

There was greater fluorescence intensity of IL-4 receptor (IL-4R) and IL-13 in the index patient compared to the healthy patient, demonstrated by immunofluorescence co-staining of IL-13 and IL-4R in the index patient and healthy control skin biopsies (FIG. 1E, FIGS. 3A-3D). There was also higher fluorescence intensity of IL-17 and IL-17Rα in the index patient compared to the healthy control, identified by co-staining of these markers (FIG. 1F, FIGS. 3E-3G), suggesting the functional signaling of the cytokines IL-13 and IL-17 at the site of itch. Furthermore, greater activation of IL-10 producing CD3⁺ T reg cells (4.90%) was found before stimulation compared to healthy controls (0.73%), the PRP control (2.58%), and the SS control (2.78%) (p<0.05) (FIG. 1G), suggesting activated immune response in the index patient. To evaluate the genetic composition of the index patient, whole genome sequencing was performed. Of all single nucleotide polymorphisms, deletions and insertions in the index patient revealed missense and intronic mutations related to IL-4, IL-13 (Table 1), IL-17 cytokines, and their signaling pathways (Table 2).

TABLE 1
Single Nucleotide Polymorphisms in genes related to IL-4 and IL-13 signaling
pathways.

| | | Variant | Variant | | | |
|---|---|---|---|---|---|---|
| Gene | Location | Classification | Type | RS ID | Ref/Alt | Impact |
| IL4R | 16:27352365 | Intron | SNP | rs3024608 | G/C | Modifier |
| IL4R | 16:27356013 | Intron | SNP | rs4787423 | C/T | Modifier |
| IL4R | 16:27359615 | Intron | DEL | rs138640688 | GTG/ [symbol] | Modifier |
| IL4R | 16:27359837 | Intron | SNP | rs3024660 | C/T | Modifier |
| IL13 | 5:132660151 | Intron | SNP | rs1295686 | T/C | Modifier |
| IL13 | 5:132660272 | Missense | SNP | rs20541 | A/G | Moderate |
| | | Mutation | | | | |
| IL13RA2 | X:115005334 | Intron | SNP | rs17095003 | G/A | Modifier |
| IL13RA1 | X:118746864 | Intron | SNP | rs144642926 | A/T | Modifier |
| IL13RA1 | X:118770512 | Intron | SNP | rs184297874 | G/A | Modifier |
| JAK2 | 9:5041690 | Intron | SNP | rs72699590 | C/G | Modifier |
| JAK2 | 9:5041692 | Intron | SNP | rs72699592 | G/T | Modifier |
| JAK2 | 9:5042046 | Intron | SNP | rs7032785 | C/T | Modifier |
| JAK2 | 9:5042082 | Intron | SNP | rs62541543 | G/A | Modifier |
| JAK2 | 9:5050706 | Silent | SNP | rs2230722 | C/T | Low |
| JAK2 | 9:5081780 | Silent | SNP | rs2230724 | G/A | Low |
| JAK2 | 9:5085417 | Intron | SNP | rs62543863 | C/T | Modifier |
| JAK2 | 9:5085859 | Intron | SNP | rs62543876 | G/C | Modifier |
| JAK2 | 9:5085929 | Intron | SNP | rs117283946 | C/G | Modifier |
| JAK2 | 9:5111225 | Intron | DEL | rs113238356 | C/ [symbol] | Modifier |
| JAK2 | 9:5111362 | Intron | DEL | rs138377711 | C/ [symbol] | Modifier |
| JAK2 | 9:5112844 | Intron | SNP | rs10815158 | G/A | Modifier |
| JAK2 | 9:5114522 | Intron | SNP | rs8884132 | G/A | Modifier |
| JAK3 | 19:17830007 | Intron | SNP | rs3212781 | C/G | Modifier |
| JAK3 | 19:17830033 | Intron | SNP | rs3212780 | C/T | Modifier |
| STAT6 | 12:57099213 | Intron | SNP | rs841718 | C/T | Modifier |
| STAT6 | 12:57099589 | Intron | SNP | rs777101090 | G/A | Modifier |

[symbol]

indicates data missing or illegible when filed

TABLE 2
Single nucleotide polymorphisms in genes related to IL-17 signaling pathways.

| | | Variant | Variant | | | |
|---|---|---|---|---|---|---|
| Gene | Location | Classification | Type | RS ID | Ref/Alt | Impact |
| IL [symbol] | [symbol] | [symbol] | SNP | [symbol] | [symbol] | [symbol] |
| IL [symbol] | [symbol] | [symbol] | SNP | [symbol] | [symbol] | Modifier |
| IL [symbol] | [symbol] | [symbol] | SNP | [symbol] | [symbol] | Modifier |
| IL [symbol] | [symbol] | [symbol] | SNP | [symbol] | [symbol] | [symbol] |
| IL [symbol] | [symbol] | [symbol] | SNP | [symbol] | [symbol] | Modifier |
| IL [symbol] | [symbol] | [symbol] | SNP | [symbol] | [symbol] | Modifier |
| IL [symbol] | [symbol] | [symbol] | SNP | [symbol] | [symbol] | [symbol] |
| IL [symbol] | [symbol] | [symbol] | SNP | [symbol] | [symbol] | Modifier |
| IL [symbol] | [symbol] | [symbol] | DEL | [symbol] | [symbol] | Modifier |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | Modifier |
| IL [symbol] | [symbol] | [symbol] | SNP | [symbol] | [symbol] | Modifier |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | DEL | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | DEL | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | [symbol] |
| IL [symbol] | [symbol] | [symbol] | SNP | [symbol] | [symbol] | Modifier |
| IL [symbol] | [symbol] | [symbol] | SNP | [symbol] | [symbol] | Modifier |
| IL [symbol] | [symbol] | [symbol] | DEL | [symbol] | [symbol] | Modifier |
| IL [symbol] | [symbol] | [symbol] | SNP | [symbol] | [symbol] | Low [symbol] |
| IL [symbol] | [symbol] | [symbol] | SNP | [symbol] | [symbol] | [symbol] |

[symbol]

indicates data missing or illegible when filed

### Increased Pathogenic IL-13 and IL-17 Producing γδT Cells in the Index Patient

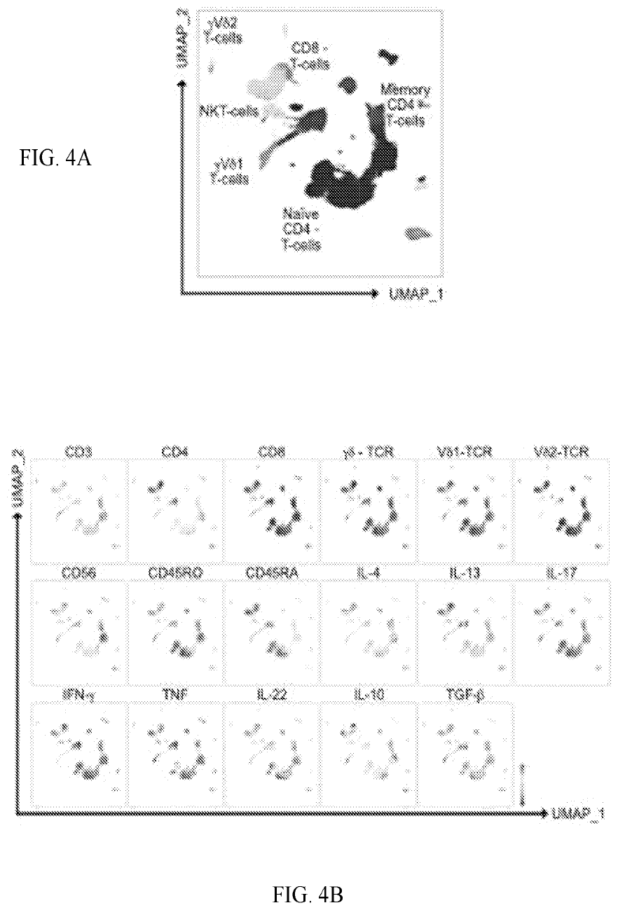

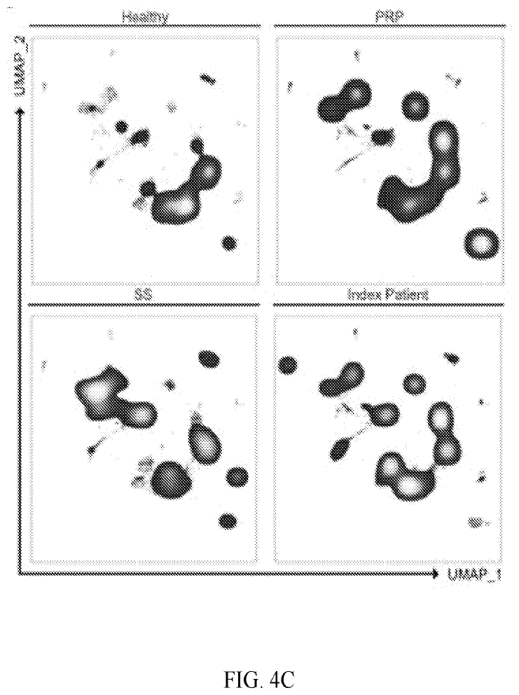

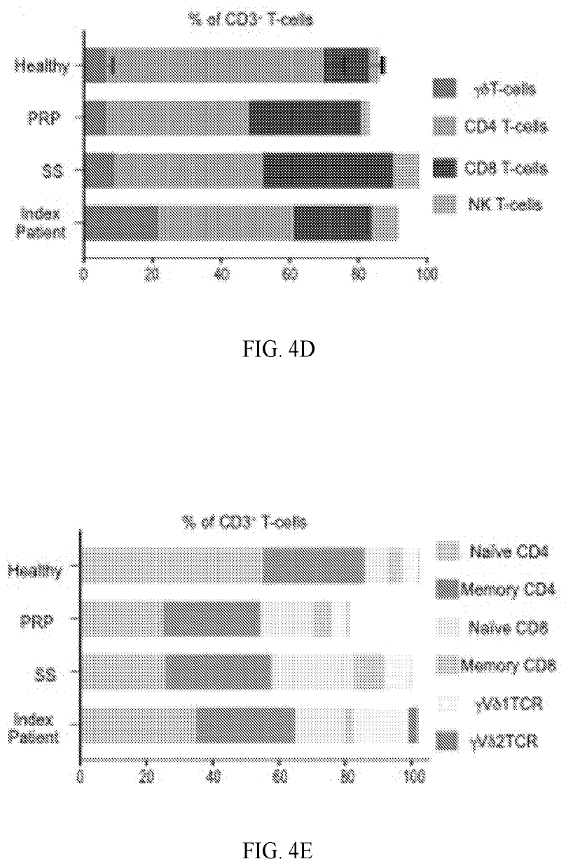

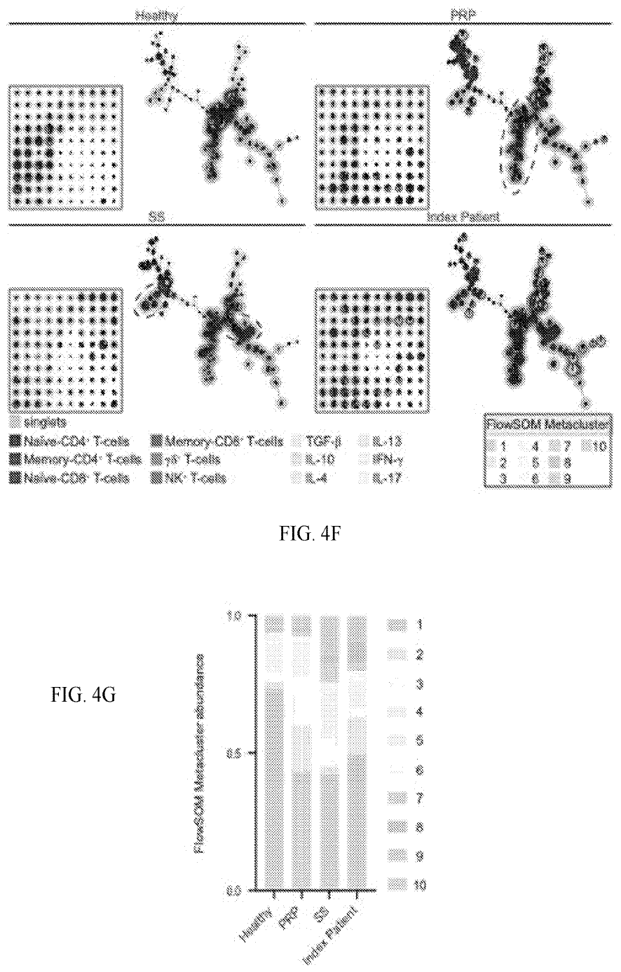

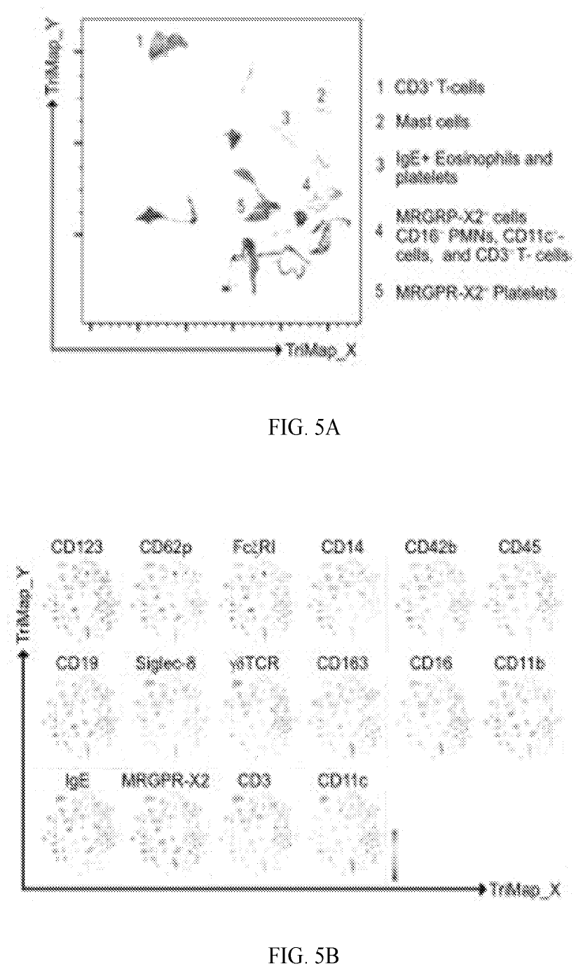

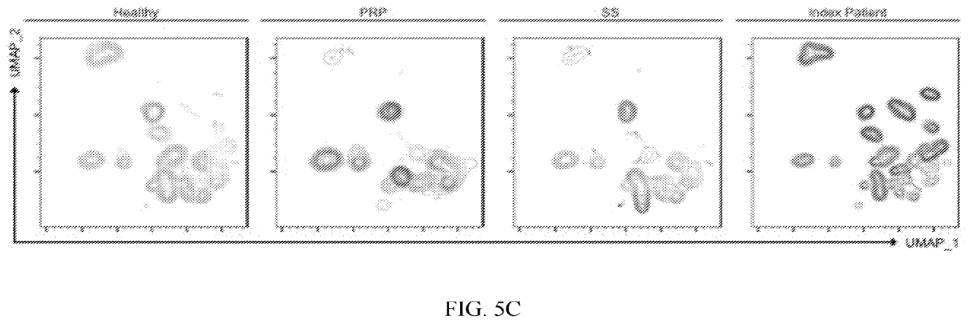

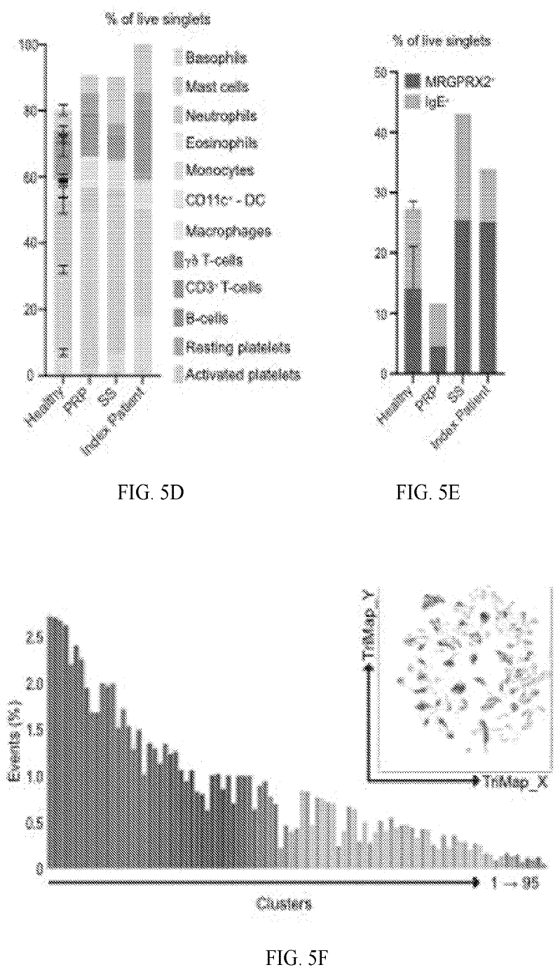

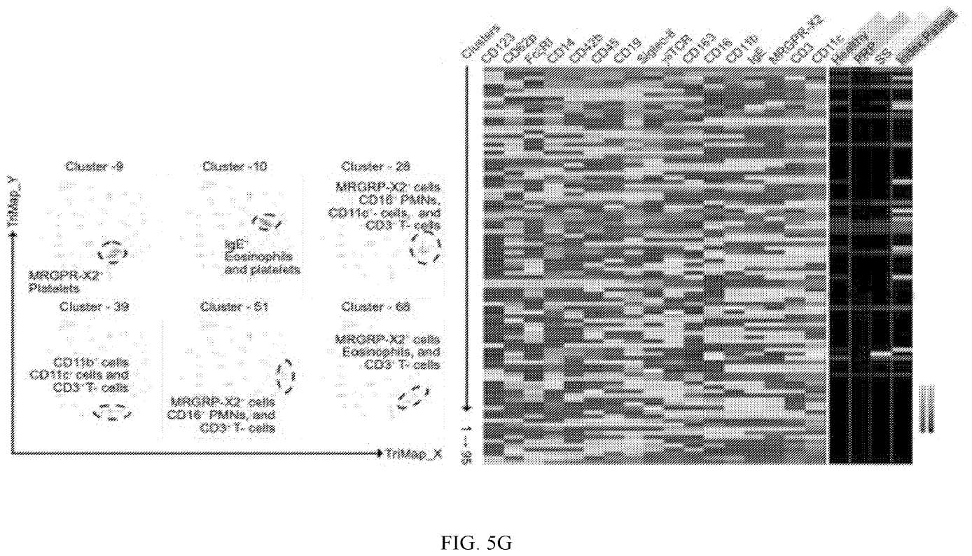

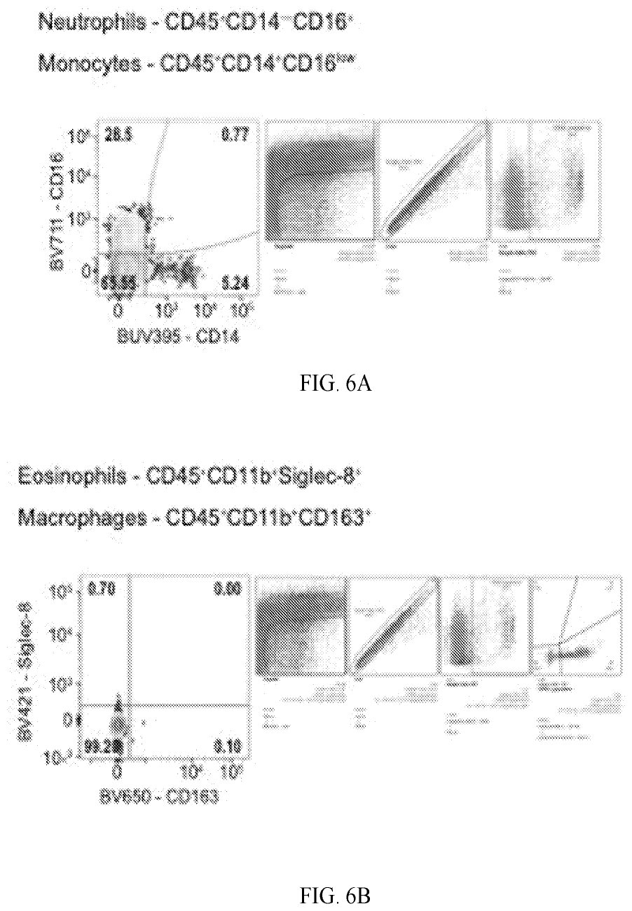

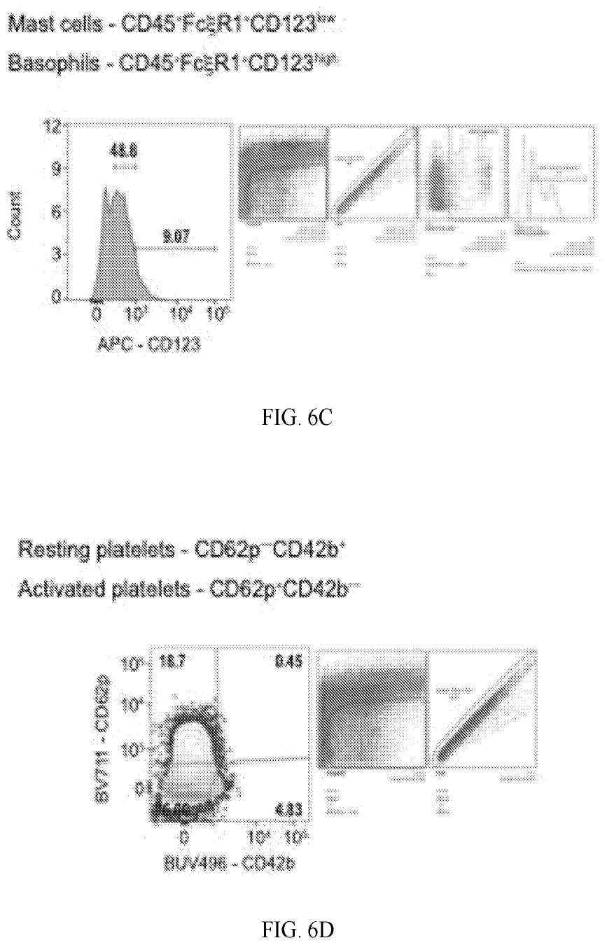

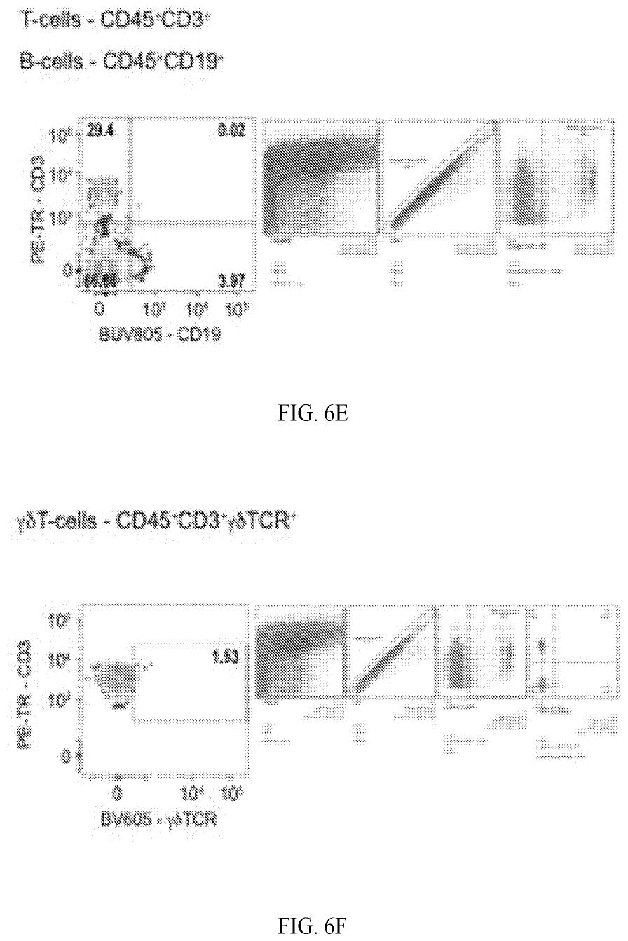

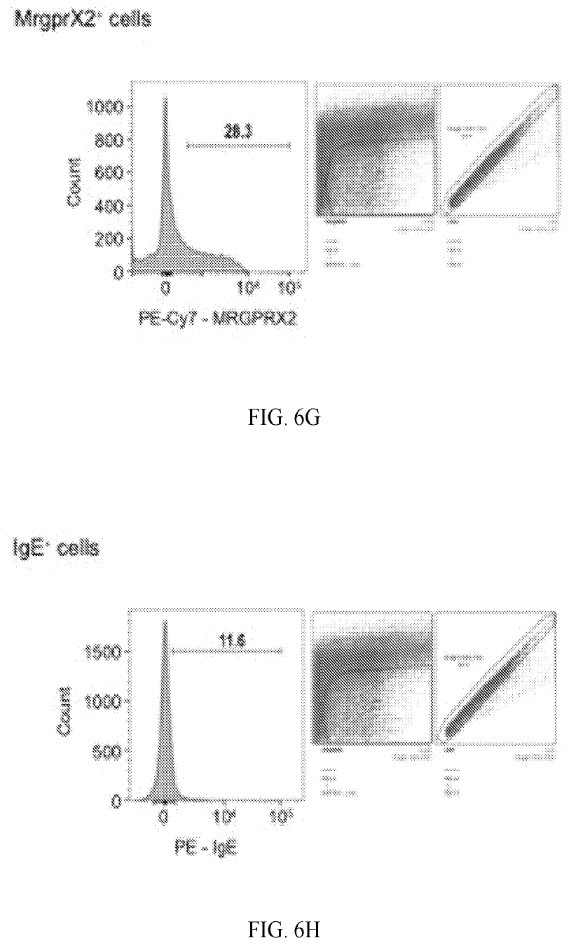

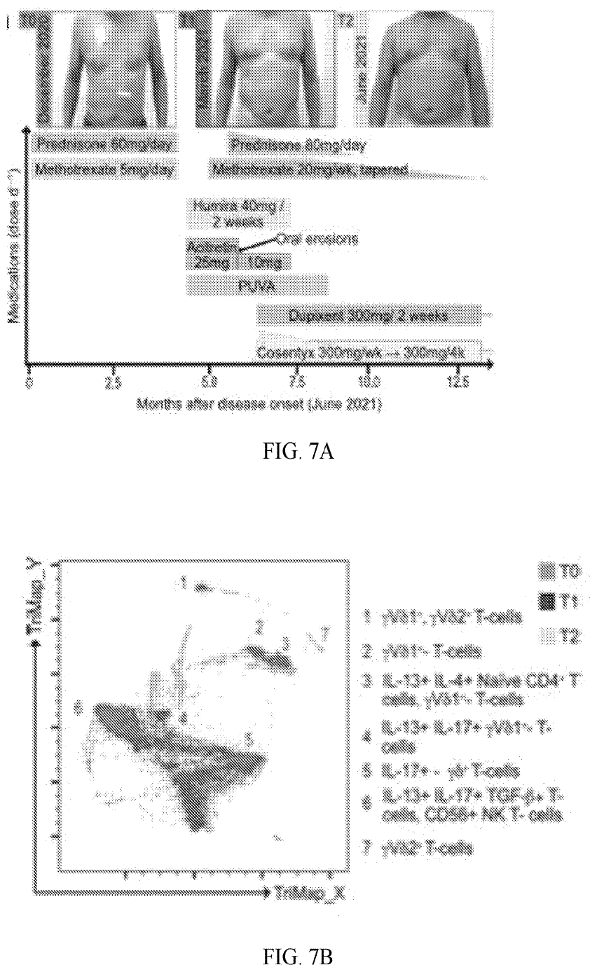

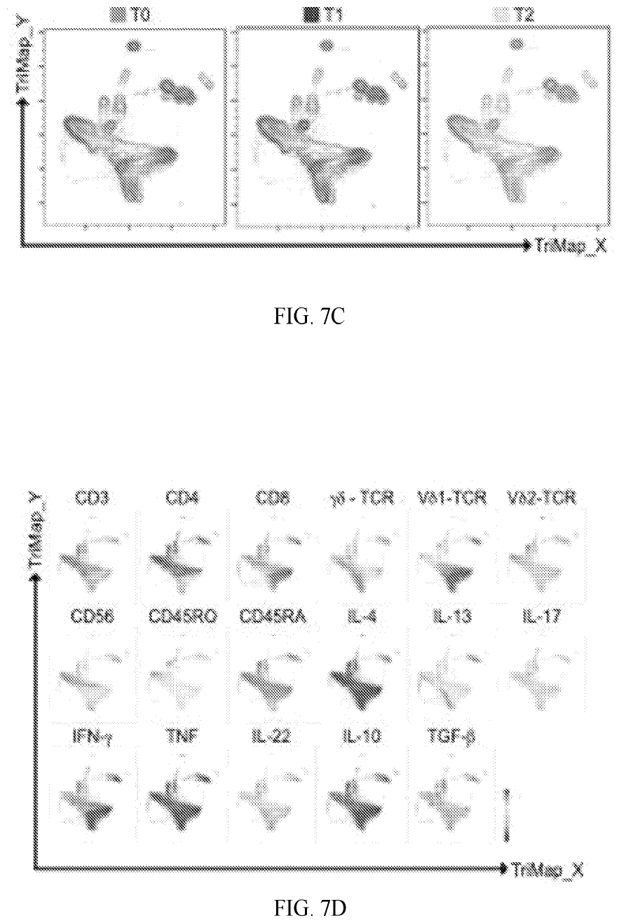

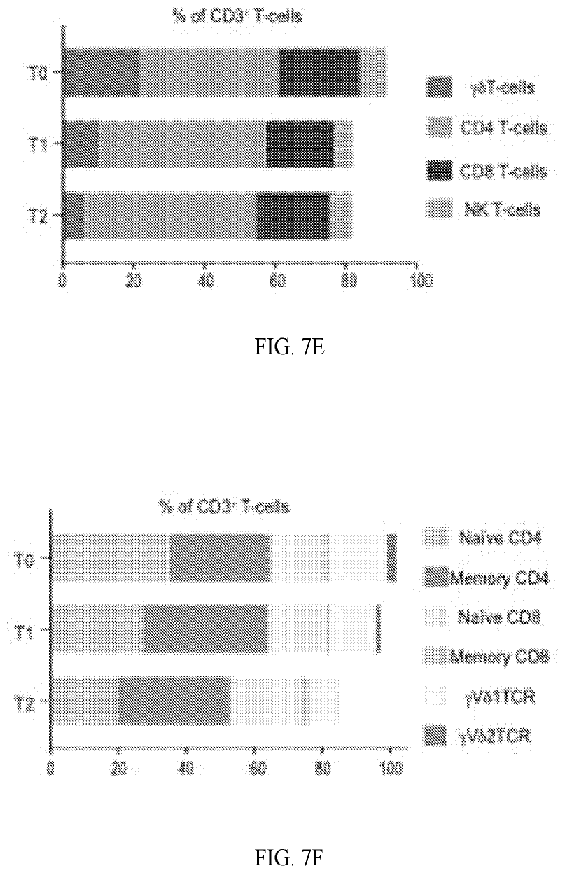

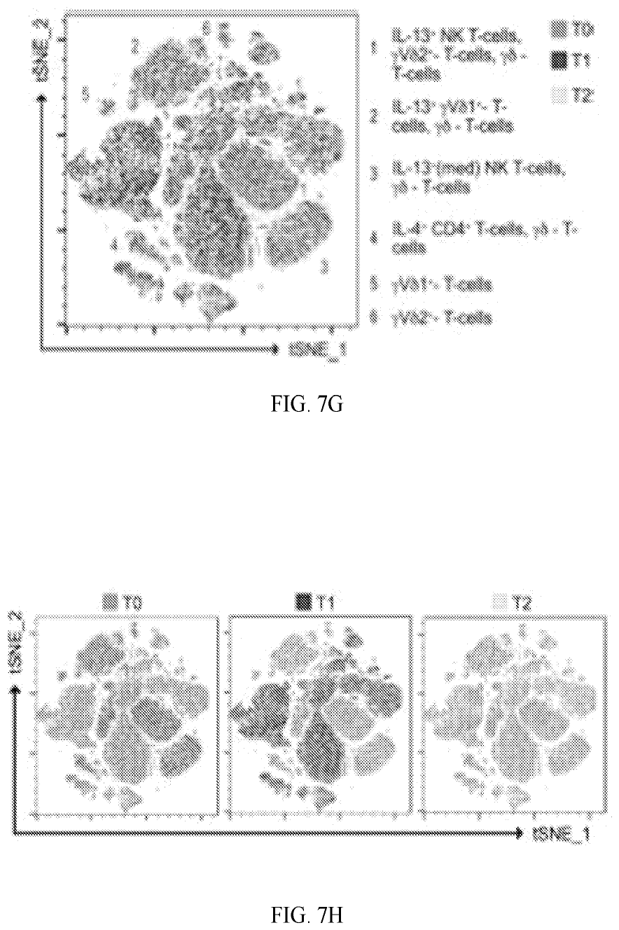

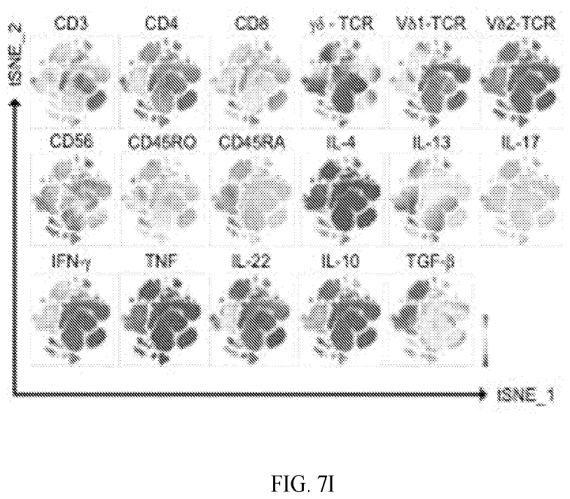

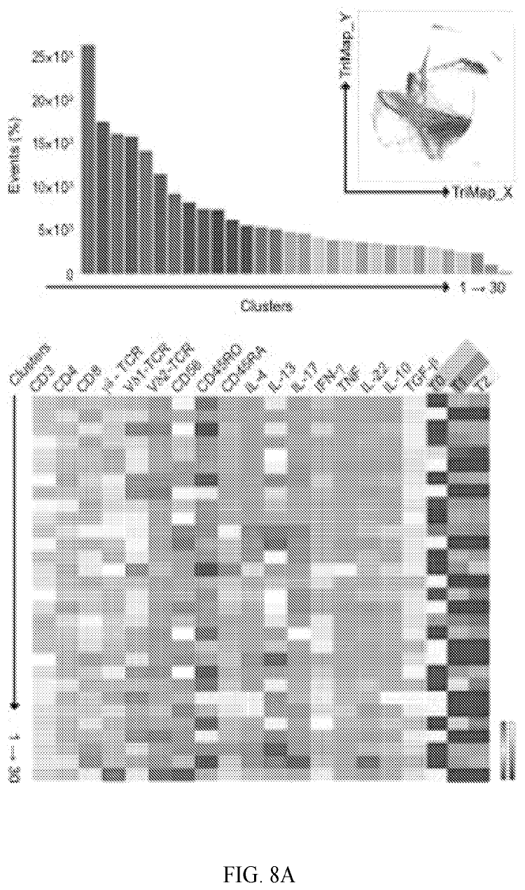

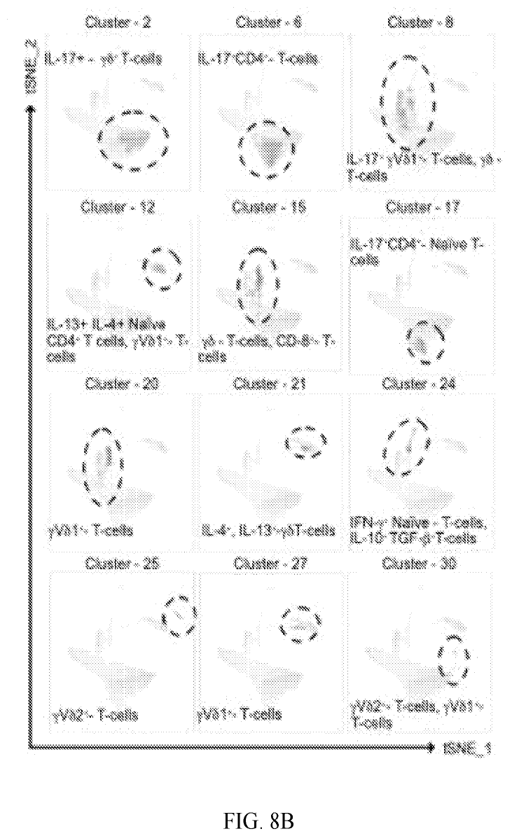

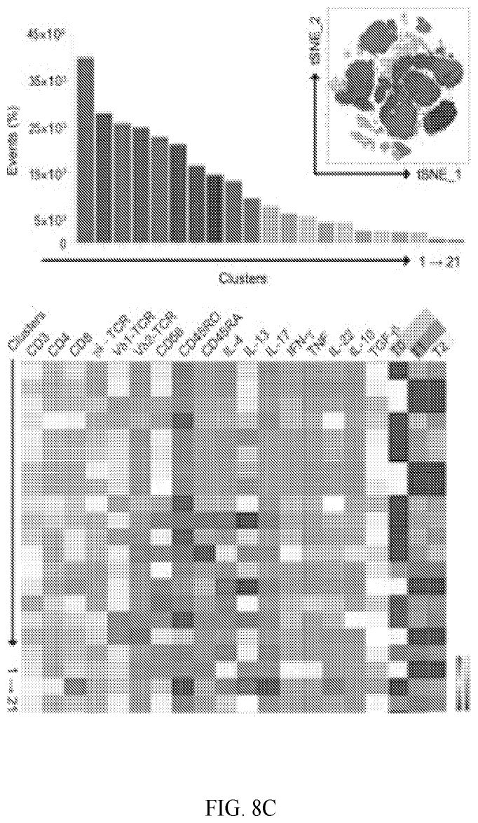

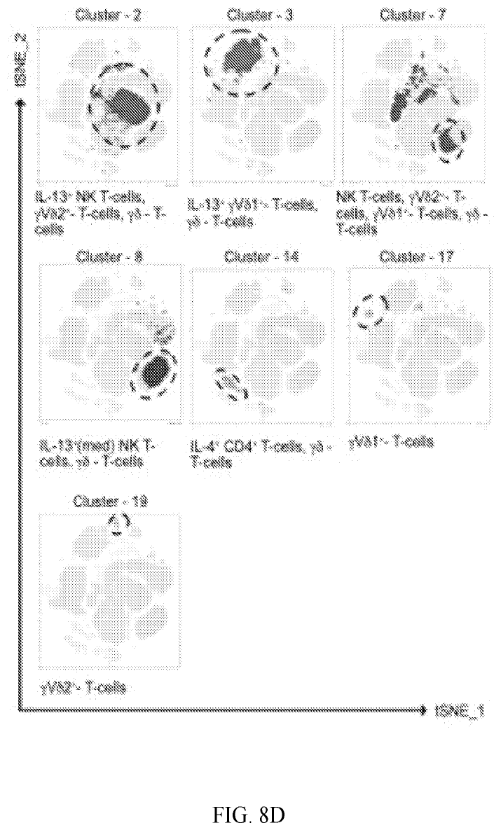

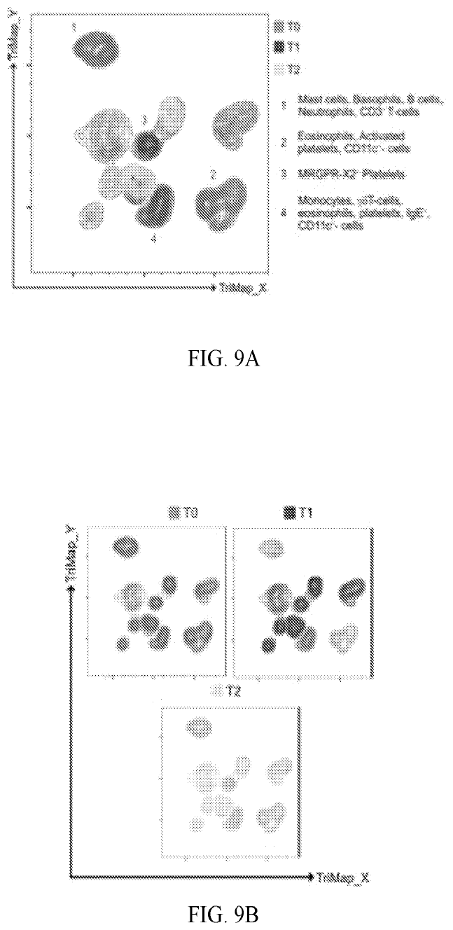

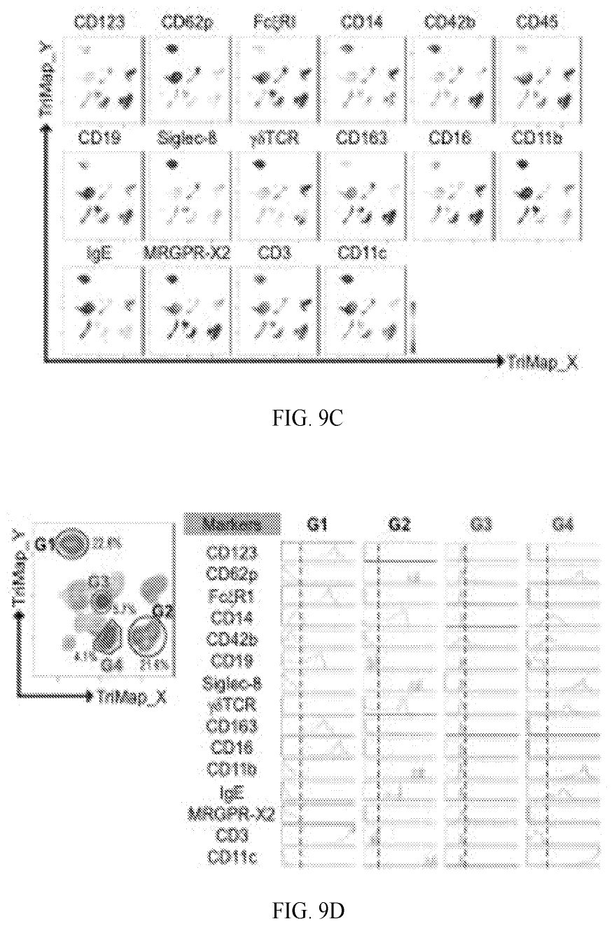

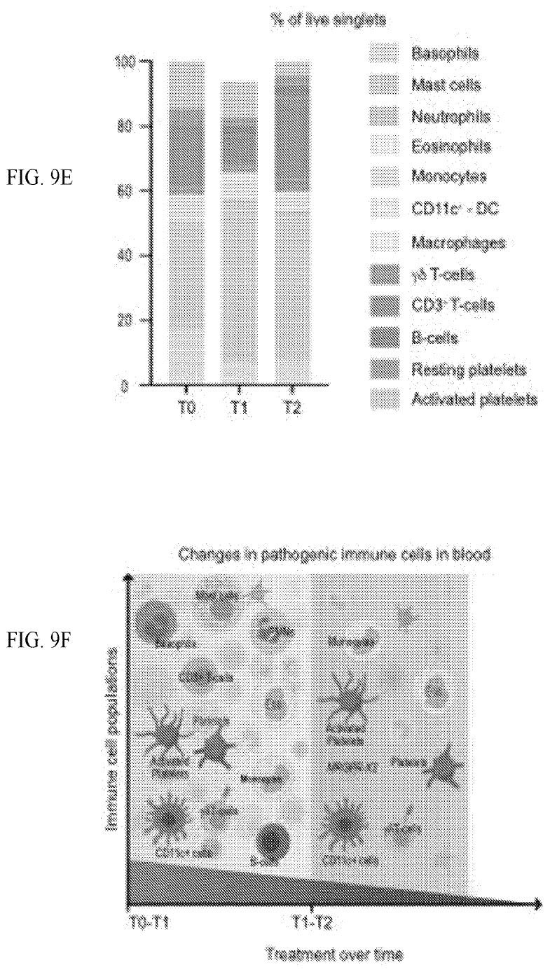

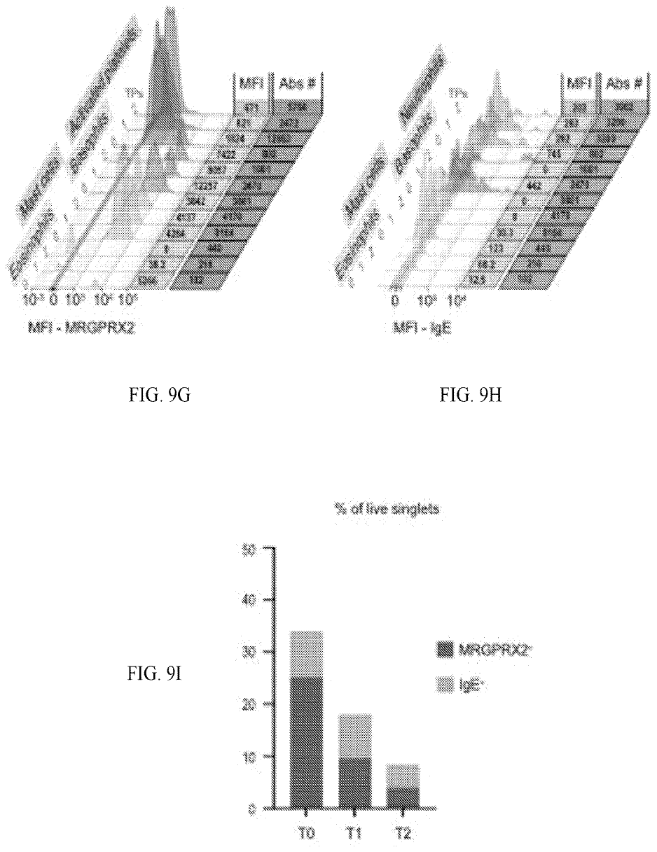

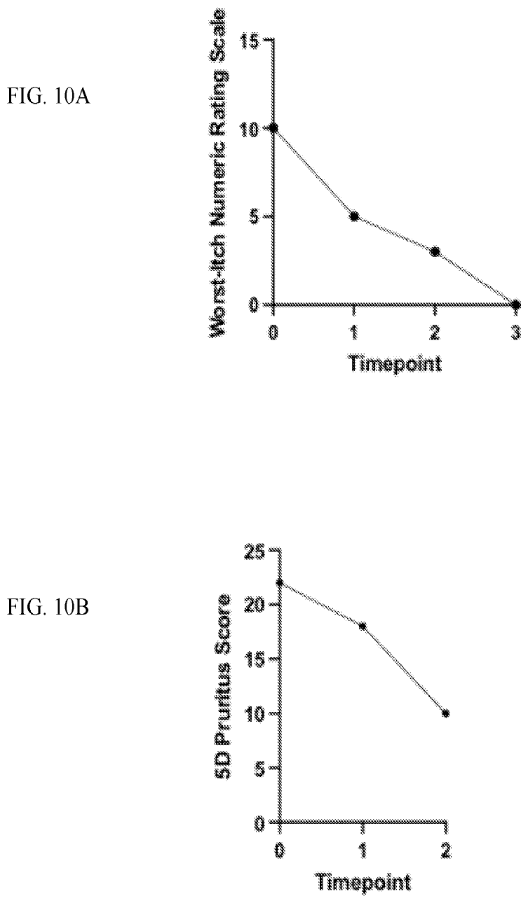

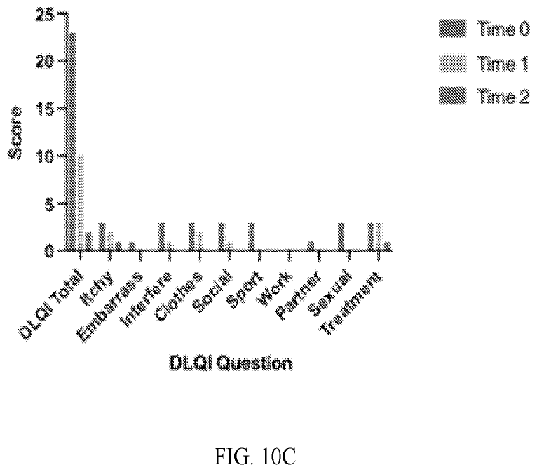

High dimensional single cell clustering algorithms, including Uniform manifold approximation and projection (UMAP) plots of T cell immune populations, were utilized to cluster subpopulations of CD3⁺ PBMCs (FIG. 4A). The individual markers in the panel used to identify these subpopulations are visualized in FIG. 4B. The index patient had increased γδT cells and a unique clonal population of γδT cells, γVδ2 T cells, compared to the healthy and erythrodermic controls; the previous analysis implicated these cells in the production of IL-13 and IL-17 in this patient (FIGS. 4C-4D), suggesting a contribution of immunopathogenic gd T cells. Breakdown of γδ T cells into subpopulations revealed higher γVδ1 and γVδ2 clonal T cell populations in the index patient; γVδ2 T cells were uniquely increased in the index patient (FIG. 4E).

Functional Immunophenotyping and Single Cell Clustering Analysis Show Distinct T Cell Profiles in Patients with Erythroderma.

To determine the similarities and differences in immune response between the index patient and other erythroderma controls, FlowSOM meta-clustering was used to cluster CD3⁺ T cell immune populations in each group, yielding 10 meta-clusters with varying abundances (FIGS. 4F-4G). Both the PRP and SS controls demonstrated similarity in some subpopulations of T cells. PRP had overlapping similarities in Cluster 2 of naive and memory CD4⁺ T cells (FIG. 4F), and SS had overlapping similarities in Cluster 9 of naive CD8⁺ T cells and Cluster 7 of CD4⁺ T-cells (FIG. 4F) with the index patient. However, there were also differences in the subpopulations and clusters between the groups, with the differences in clusters including Clusters 4, 5, and 6 with gd T cells and NK-T cells were different in the index patient (FIGS. 4F-4G). These data suggest that functional immunophenotyping and single cell clustering analysis from PBMCs can identify distinct T cell profiles in erythroderma patients.

### Increased Basophils and MRGPRX2 Expressing Activated Platelets in the Index Patient

Immunophenotyping of granulocytic and monocytic immune cell populations by FACS of whole blood from the index patient, erythrodermic controls, and healthy controls revealed increased basophils and unique IgE- and MRGPRX2-expressing immune populations in the index patient (FIGS. 5A-5B). Differences in granulocytic and monocytic cell populations in the index patient were identified by single cell clustering of the whole blood cell populations via UMAP. Unique IgE-expressing eosinophils, CD16⁺ neutrophils (PMNs), CD11c+ cells and MRGPRX2-expressing platelets were present in the index patient (FIG. 5C). Furthermore, activated platelets were elevated in the index patient and the SS erythrodermic control, and basophils were elevated in the index patient compared to other groups (FIG. 5D). The index patient and SS control also had higher proportions of MRGPRX2-expressing immune populations than the healthy controls or PRP control (FIG. 5E). There were 95 clusters of immune cells from whole blood, with varying clustering signatures present in each patient group, identified by single-cell clustering (FIG. 5F). Finally, there were unique IgE-expressing and MRGPRX2-expressing populations in the index patient, depicted via the TriMAP plot of the clusters (FIGS. 5F-5G). The gating strategy for whole blood FACS is depicted in FIG. 6.

### Dual Inhibition of IL-4RA and IL-17RA Show Reversal of Immunopathogenic Populations

The index patient was treated with numerous systemic medications, including immunosuppressants and antimetabolites, with minimal response. He was then started on dupilumab, a monoclonal antibody inhibitor of the IL-4R alpha subunit, which blocks both IL-4 and IL-13 signaling, and secukinumab, a monoclonal antibody inhibitor of IL-17A, preventing its interaction with IL-17R. FIG. 7A demonstrates the clinical and treatment course of the index patient through three-time intervals. Along with clinical improvement, there was also an improvement in quality-of-life indicators in this patient. Notably, there was a 10-point decrease in itch overall. The patient's 5-dimension pruritus score decreased from very severe to mild pruritus, and their dermatology life quality index dropped from having an extremely large effect on their life to a moderate impact (FIGS. 10A-10C).

To evaluate the treatment regimen, PBMCs, and whole blood at all three-time intervals from the index patient were collected for analysis. The changes in immune responses, including T cells, granulocytes, and monocytes were followed for as outlined in FIG. 1B. Stimulated PBMCs of CD3⁺ T cells in all three-time intervals showed alterations in immune cell populations between the treatment course, as shown by the TriMAP plot (FIG. 7B). When stratified by time interval, there were changes in the makeup of CD3⁺ cells in this patient, identified by specific cell-specific markers (FIGS. 7C-7D). Breakdown of the cell types demonstrated a decrease in γδ T cells, specifically clearance of pathogenic γVδ2 T cells, which produce IL-13 and IL-17 in this patient. There was also decreased natural killer (NK) T cells (FIGS. 7E-7F). Additionally, there was reduced expression of IL-17⁺ γδT cells and IL-13*/IL-4⁺ naive γδ T cells or memory T cells with treatment, identified by single cell clustering analysis in stimulated PBMCs by time intervals (FIGS. 8A-8D).

There was clearance of NK T cells with treatment, as visualized by T-distributed stochastic neighbor embedding (tSNE) plots of unstimulated T cells were also stratified by specific markers. Additionally, there was a reduction of IL-13 producing immune cell populations (FIG. 7D), including NK T cells and γVδ1 T cells in unstimulated PBMCs (FIG. 7D), identified via single cell clustering analysis in unstimulated PBMCs by time intervals.

Similarly, there was a reversal of immunopathogenic populations after dual inhibition of IL-4RA and IL-17Rα pathways, revealed by changes in the index patient's whole blood populations at three intervals. All immune populations from the index patient (FIG. 9A) were stratified by those present at each time interval (FIG. 9B), demonstrating immune populations that were cleared after treatment. There were four unique clusters in these data, as identified by the differential expression of each immune marker (FIG. 9C), and single cell cluster analysis revealed four unique clusters (FIG. 9D), which are highlighted in FIG. 9A. There was also a decrease in the percentages of basophils and activated platelets in the index patient after treatment, demonstrated by analysis of the granulocytic and monocytic cell subtypes at each time interval. The changes in immunopathogenic populations during the treatment interval by dual inhibitors IL-4Rα and IL-17Rα in the index patient are graphically represented in FIG. 9F.

### Regulation of MRGPRX2 by Dual Inhibition of IL-4RA and IL-17RA

It was found that the expression of MRGPRX2 was regulated in immune cells with dual inhibition of IL-4Rα and IL-17Rα. Quantification of mean fluorescence intensity (MFI) of MRGPRX2-expressing cells at each time interval revealed decreased MFI after treatment (FIG. 9G). This reduced expression of the receptor was independent of the immune cell numbers, evidently in eosinophils, as shown by the absolute count (FIG. 9G). Furthermore, quantification of IgE+ cells at each time interval did not reveal any definite pattern in IgE, as shown by the MFI and absolute cell counts (FIG. 9H). These data suggest that this unique form of erythroderma is characteristic of MRGPRX2 expression in immune cells (FIG. 9I), and the percentage of all MRGPRX2-expressing cells decreases substantially with treatment, suggesting that, unlike IgE, MRGPRX2 expression is regulated with treatment with IL-13 and IL-17 inhibition.

Together, these results demonstrate that the presence of (a) an elevated level of IL-13 signaling, (b) an elevated level of IL-17 signaling, and/or (c) an elevated level of a MRGPRX2 polypeptide in a sample (e.g., a blood sample containing one or more PMBCs) from a mammal having an inflammatory skin disease (e.g., erythroderma such as refractory erythroderma) can be used to identify that mammal as being likely to respond to a particular treatment (e.g., a particular targeted therapy such as one or more inhibitors of the IL-13 signaling pathway (e.g., dupilumab) and/or one or more inhibitors of the IL-17 signaling pathway (e.g., secukinumab)).

### Methods

### Index Patient

The index patient provided written consent to protocols, which enabled de-identified research use of biospecimens, genetic testing results, clinical data, and standard of care.

### Sampling Human Materials

Lesional skin biopsy was obtained from the patient, two erythrodermic controls, and three healthy control patients by two 4 mm biopsies after local anesthesia with lidocaine. PBMCs were obtained from fresh blood samples from the patient and controls. Plasma was also obtained from fresh blood samples from the patient and controls.

### Cell Isolation and Processing.

PBMCs. 1 mL of WB was removed, 100 μL DMSO was added and cryopreserved using a slow freeze at −80 °C, transferred to LN2 vapor phase 24-72 hours later. The remaining blood was used for ficoll gradient, spun at 630×g for 25 minutes. Additional ficoll spin were performed to clean up samples with severe hemolysis. PBMC layer was removed, washed using DPBS, and counted using Vi-Cell counter. Wash spin and post count spin were centrifuged at 315×g for 5 minutes. PBMCs were frozen down at ~5e6 cells/vial using RPMI+10% FBS+10% DMSO freezing media. Samples were placed in a slow freeze at −80 °C, transferred to LN2 vapor phase 24-72 hours later.

Plasma. Plasma was centrifuged at 1960×g for 10 minutes, and was then transferred (avoiding buffy layer) to create up to ×4 1 mL aliquots. Samples were then placed in a slow freeze at −80 °C, and transferred to LN2 vapor phase 24-72 hours later.

Whole Blood. To freeze whole blood, DMSO was added to the sample (10% by volume) to inhibit ice-crystal formation inside the cells and freeze the samples at a controlled rate of 1 °C/hour. The storage temperature for whole blood was −180 °C.

Storage conditions. Samples were frozen down −1 °C/minute in CoolCells and then transferred to liquid nitrogen vapor phase after 24 hours. All inventory was stored in liquid nitrogen vapor phase (−180 °C) until they were retrieved.

### PBMC Flow Cytometry

Viable cells (about 3-5×10⁶ cells) were stimulated in RPMI with eBioscience Cell Stimulation Cocktail. Cells were collected after stimulation, and single-cell suspensions were obtained after filtering through a 40-mm cell filter. The cells were then washed in RPMI. The single-cell suspension was incubated with TruStain fcX (BioLegend, San Diego, CA) to block Fc receptor binding and was resuspended to label with mAbs against extracellular cell surface markers. The cell surface markers were incubated with the cells in Hanks Balanced Salt Solution with 2% Calf Serum and 5 mM 4-(2-hydroxyethyl)-1-piperazineethanesulfonic acid along with Brilliant Stain Buffer (BD Biosciences, San Jose, CA). The stained cells were washed with PBS and stained for viability (Zombie Aqua Fixable Viability Kit, BioLegend). The surface-labeled cells were fixed in the BD Cytofix/Cytoperm Buffer kit (BD Biosciences). The cells were further labeled for intracellular cytokine markers. The mAb-labeled cells were then washed in intracellular staining buffer and resuspended in Stabilizing Fixative (BD Biosciences). Cell acquisition was performed on the BD LSRFortessa flow cytometer (BD Biosciences), and data were analyzed using Cytobank software (Cytobank, Mountain View, CA) and FlowJo 10 and R Statistical program V4.0. Samples were normalized with R statistical program scripts with CytoNorm v.1.23 and DownSample v3. Data on a minimum of 50000 events in the live singlets gate were collected and analyzed. Single cell analysis was performed with R statistical program scripts with Cluster algorithms including Phenograph v3, TriMap v0.2 and UMAP v3.2. The absolute number of the corresponding cell population was calculated as the total number of live cells % of the corresponding cell population/100. For immunophenotyping of the T cell subsets, including CD4, CD8, gd, and invariant NK T cells, the cells were first gated on live cells, singlets and CD3φ cells, CD4φCD8- (T helper) cells, CD8φCD4- (cytotoxic T) cells, CD4-CD8-γδTCRφ (γδ T) cells, and CD4-CD8-CD56φ (invariant NK T) cells

### Whole Blood Flow Cytometry

Cryopreserved whole blood (1 mL) samples were thawed on ice with 1 ml ACK lysis buffer (Quality Biological) for 7 minutes. Samples were washed in PBS and transferred to a 96-well U-bottom plate for staining. Samples were stained with LIVE/DEAD Fixable Dead Cell Stain (Zombie-Aqua, Thermo Fisher, L34957) at room temperature for 20 minutes in PBS. Samples were washed and stained with antibodies for 20 minutes on ice in FACS Staining Buffer along with Brilliant stain buffer 50 μL. Human Fc block was added to the surface stain master mix. The cells were then fixed with BD Cytofix. Flow cytometry was performed on BD Fortessa and data were analyzed using FlowJo 10 and R Statistical program V4.0. Samples were normalized with R statistical program scripts with CytoNorm v.1.23 and DownSample v3. Data on a minimum of 50000 events in the live singlets gate were collected and analyzed. Single cell analysis was performed with R statistical program scripts with Cluster algorithms including Phenograph v3, TriMap v0.2 and UMAP v3.2.

### Immunofluorescence Staining

Skin punch biopsy specimens from each participant were fixed in neutral-buffered 10% formalin. The specimens were then embedded in paraffin and cut into 5-μm sections, which were placed on slides. Slides were submerged in pre-heated antigen retrieval solution (Cell Marque, 920P-06) and microwaved until boiling for 15 minutes, after which they were washed with PBS three times and samples were treated with blocking reagent (Dako, X0909) for 1 hour at room temperature in a humidified, light-protected chamber. Samples were then stained with primary antibodies (IL-4R, 1:80, LifeSpan BioSciences, LS-C11209 and IL-13, 1:200, LifeSpan BioSciences, LS-B7417; IL-17A, 1:100, Abcam, ab189377 and IL-17RA, 1:200, LifeSpan BioSciences, LS-B15779) and incubated at 4 °C overnight. The next day, samples were washed with PBST three times, stained with secondary antibodies (Alexa Fluor 488, 1:500, Abcam, ab150077 and Alexa Fluor 568, 1:500, Invitrogen, A-11031) for 1 hour at room temperature, and washed again with PBST. Slides were counterstained with DAPI working solution for 30 minutes, rinsed with PBS, and mounted with ProLong Diamond (Invitrogen, P36970). All images were taken with a Leica SP8 confocal microscope and analyzed using ImageJ (National Institutes of Health).

### Whole Genome Sequencing

Whole genome analysis was conducted on PBMCs from the index patient's middle time interval. DNA from the sample was extracted using Qiagen's DNeasy kit. The library preparation was performed using the Agilent SureSelectXT Library Prep Kit/SureSelectXT Target V5 kit and was enriched using the Enrichment System for Illumina Version B.2, and subsequently was sequenced using NovaSeq6000 S4 (150 bp PE). The bwa.v0.7.7 tool was used for running the read alignments against the hg38 genome, and Piccard-tools1.119 was used to add read groups and remove duplicate reads. GATK v3.6.0 base call recalibration steps were used to create a final alignment file. The bcftools v1.5 was used to call variants against the reference. MuTect2 v3.6.0 was used to call variants against a panel of normals, and snpEFF (v4.1) was used to annotate the passed variant calls. Subsequently, the passed variants were converted to Mutation Annotation Format (MAF) using vcf2maf-1.6.19. Somatic variants in the patient that intersected with major IL-4, IL-13, and IL-17 pathways were reported, and Integrative Genomics Viewer (IGV) was used to view individual variants.

### Example 2: Treating Erythroderma

A biological sample containing PMBCs (e.g., a blood sample such as a whole blood sample or a plasma sample) is obtained from a human having erythroderma. The obtained sample is examined for the presence or absence of (a) an elevated level of IL-13 signaling, (b) an elevated level of IL-17 signaling, and/or (c) an elevated level of a MRGPRX2 polypeptide. In some cases, PBMC flow cytometry is performed to detect the presence or absence of (a) an elevated level of IL-13 signaling, (b) an elevated level of IL-17 signaling, and/or (c) an elevated level of a MRGPRX2 polypeptide. If the presence of (a) an elevated level of IL-13 signaling, (b) an elevated level of IL-17 signaling, and/or (c) an elevated level of a MRGPRX2 polypeptide is detected in the sample, as compared to a control level, then the human is administered one or more inhibitors of the IL-13 signaling pathway (e.g., dupilumab) and/or one or more inhibitors of the IL-17 signaling pathway (e.g., secukinumab). The administered inhibitor(s) can reduce the severity of one or more symptoms of the erythroderma (e.g., pruritus (itchy skin), skin redness, skin inflammation, and/or skin pain).

### Example 3: Treating Erythroderma

A human having erythroderma and identified as having the presence of (a) an elevated level of IL-13 signaling, (b) an elevated level of IL-17 signaling, and/or (c) an elevated level of a MRGPRX2 polypeptide is administered one or more inhibitors of the IL-13 signaling pathway (e.g., dupilumab) and/or one or more inhibitors of the IL-17 signaling pathway (e.g., secukinumab). The administered inhibitor(s) can reduce the severity of one or more symptoms of the erythroderma (e.g., pruritus (itchy skin), skin redness, skin inflammation, and/or skin pain).

### Example 4: Treating Erythroderma

A biological sample containing PMBCs (e.g., a blood sample such as a whole blood sample or a plasma sample) is obtained from a human having erythroderma. The obtained sample is examined for the presence or absence of (a) an elevated level of IL-13 signaling, (b) an elevated level of IL-17 signaling, and/or (c) an elevated level of a MRGPRX2 polypeptide. In some cases, PBMC flow cytometry is performed to detect the presence or absence of (a) an elevated level of IL-13 signaling, (b) an elevated level of IL-17 signaling, and/or (c) an elevated level of a MRGPRX2 polypeptide. If the absence of (a) an elevated level of IL-13 signaling, (b) an elevated level of IL-17 signaling, and/or (c) an elevated level of a MRGPRX2 polypeptide is detected (e.g., if the presence of (a) an elevated level of IL-13 signaling, (b) an elevated level of IL-17 signaling, and (c) an elevated level of a MRGPRX2 polypeptide is not detected) in the sample, as compared to a control level, then the human is administered one or more treatments that are not an inhibitor of the IL-13 signaling pathway or an inhibitor of the IL-17 signaling pathway (e.g., prednisone, acitretin, methotrexate, cyclosporine, azathioprine, mycophenolate mofetil, adalimumab, ixekizunab, risakizumab, guselkumab, certolizumab, illumya, ixekizumab, brodalumab, nemolizumab, abrocitinib, upadacitinib, apremilast, and combinations thereof). The administered inhibitor(s) can reduce the severity of one or more symptoms of the erythroderma (e.g., pruritus (itchy skin), skin redness, skin inflammation, and/or skin pain).

### Example 5: Treating Erythroderma

A human having erythroderma and identified as lacking the presence of (a) an elevated level of IL-13 signaling, (b) an elevated level of IL-17 signaling, and (c) an elevated level of a MRGPRX2 polypeptide is administered one or more treatments that are not an inhibitor of the IL-13 signaling pathway or an inhibitor of the IL-17 signaling pathway (e.g., prednisone, acitretin, methotrexate, cyclosporine, azathioprine, mycophenolate mofetil, adalimumab, ixekizunab, risakizumab, guselkumab, certolizumab, illumya, ixekizumab, brodalumab, nemolizumab, abrocitinib, upadacitinib, apremilast, and combinations thereof). The administered inhibitor(s) can reduce the severity of one or more symptoms of the erythroderma (e.g., pruritus (itchy skin), skin redness, skin inflammation, and/or skin pain).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-7. (canceled)

8. A method for treating a mammal having an inflammatory skin disease, said method comprising:
determining that a sample obtained from said mammal comprises the presence of: (a) an elevated level of IL-13 signaling, (b) an elevated level of IL-17 signaling, and (c) an elevated level of a MRGPRX2 polypeptide; and
administering an inhibitor of an IL-13 signaling pathway or an inhibitor of an IL-17 signaling pathway to said mammal.

9. A method for treating a mammal having an inflammatory skin disease, and identified as having the presence of: (a) an elevated level of IL-13 signaling, (b) an elevated level of IL-17 signaling, and (c) an elevated level of a MRGPRX2 polypeptide, said method comprising:
administering an inhibitor of an IL-13 signaling pathway or an inhibitor of an IL-17 signaling pathway to said mammal.

10. The method of claim 8, wherein said mammal is a human.

11. The method of claim 8, wherein said inflammatory skin disease is erythroderma.

12. The method of claim 11, wherein said erythroderma is a refractory erythroderma.

13. The method of claim 8, wherein said inhibitor of said IL-13 signaling pathway is selected from the group consisting of dupilumab, tralokinumab, lebrikizumab, and eblasakinumab.

14. The method of claim 8, wherein said inhibitor of said IL-17 signaling pathway is secukinumab.

15. The method of claim 8, wherein said administering comprising administering to said mammal dupilumab and secukinumab.

16. A method for treating a mammal having an inflammatory skin disease, said method comprising:
determining that a sample obtained from said mammal lacks the presence of: (a) an elevated level of IL-13 signaling, (b) an elevated level of IL-17 signaling, and (c) an elevated level of a MRGPRX2 polypeptide; and
administering an alternative treatment to said mammal, wherein said alternative treatment is not an inhibitor of an IL-13 signaling pathway or an inhibitor of an IL-17 signaling pathway.

17. A method for treating a mammal having an inflammatory skin disease, and identified as lacking the presence of: (a) an elevated level of IL-13 signaling, (b) an elevated level of IL-17 signaling, and (c) an elevated level of a MRGPRX2 polypeptide, said method comprising:
administering an alternative treatment to said mammal, wherein said alternative treatment is not an inhibitor of an IL-13 signaling pathway or an inhibitor of an IL-17 signaling pathway.

18. The method of claim 16, wherein said mammal is a human.

19. The method of claim 16, wherein said inflammatory skin disease is erythroderma.

20. The method of claim 16, wherein said alternative treatment comprises administering prednisone, acitretin, methotrexate, cyclosporine, azathioprine, mycophenolate mofetil, adalimumab, ixekizunab, risakizumab, guselkumab, certolizumab, illumya, ixekizumab, brodalumab, nemolizumab, abrocitinib, upadacitinib, and/or apremilast to said mammal.

21-23. (canceled)

24. The method of claim 9, wherein said mammal is a human.

25. The method of claim 9, wherein said inflammatory skin disease is erythroderma.

26. The method of claim 25, wherein said erythroderma is a refractory erythroderma.

27. The method of claim 9, wherein said inhibitor of said IL-13 signaling pathway is selected from the group consisting of dupilumab, tralokinumab, lebrikizumab, and eblasakinumab.

28. The method of claim 9, wherein said inhibitor of said IL-17 signaling pathway is secukinumab.

29. The method of claim 9, wherein said administering comprising administering to said mammal dupilumab and secukinumab.
